(19) United States
(12) Patent Application Publication
(10) Pub. No.: US 2021/0346391 A1
(43) Pub. Date: Nov. 11, 2021

(54) TREATMENT OF PRURITUS WITH P2X3 MODULATORS

(71) Applicant: Bellus Health Cough Inc., Laval (CA)

(72) Inventors: Antonios MATZOURANIS, Laval (CA); Nathalie CHAURET, Laval (CA); Denis GARCEAU, Laval (CA)

(21) Appl. No.: 17/283,904

(22) PCT Filed: Oct. 9, 2019
(86) PCT No.: PCT/IB2019/001122

§ 371 Date: Apr. 8, 2021

Related U.S. Application Data
(60) Provisional application No. 62/744,006, filed on Oct. 10, 2018.

(51) Int. Cl. A61K 31/5377; A61P 17/04; A61K 31/4545; A61K 31/437; A61K 31/496; A61K 31/4035; A61K 31/439; A61K 31/438; A61K 31/495; A61K 31/454; A61K 31/451

(52) U.S. Cl. CPC A61K 31/5377; A61P 17/04; A61K 31/4545; A61K 31/437; A61K 31/496; A61K 31/451; A61K 31/439; A61K 31/438; A61K 31/495; A61K 31/454; A61K 31/4035

(57) ABSTRACT

Methods of treating pruritus in a mammal with a P2X3 antagonist are disclosed. Said P2X3 antagonist is preferably a compound of Formula (I). Said pruritus may be associated with an inflammatory skin disorder, an infectious skin disease, an autoimmune skin disease or a pregnancy-related skin disease. The P2X3 antagonist may be administered by intravenous administration, subcutaneous administration, oral administration, inhalation, nasal administration, topical administration or ophthalmic administration and may be used in conjunction with a NK-1 antagonist. The P2X3 antagonist acts by inhibiting pathological ATP release associated with hyperexcitability of afferent pruriceptive neurons, thus dampening peripheral hypersensitivity to itch via broad mechanism independent on the pathological stimuli acting at itch receptors.

### CROSS-REFERENCE

This application claims benefit of U.S. Provisional Application No. 62/744,006, filed on Oct. 10, 2018, which is herein incorporated by reference in its entirety.

### BACKGROUND

Pruritus is defined as an unpleasant sensation that provokes the desire to scratch. Pruritus may be localized or generalized and can occur as an acute or chronic condition. Certain systemic diseases have long been known to cause pruritus that ranges in intensity from a mild annoyance to an intractable, disabling condition which can be a diagnostic and therapeutic challenge.

### BRIEF SUMMARY OF THE INVENTION

This disclosure provides, for example, methods of treating pruritus in a mammal with a P2X3 modulator. The disclosure also provides for the use of P2X3 modulators as medicaments and/or in the manufacture of medicaments for treating pruritus in mammals, such as humans. In some embodiments, the P2X3 modulator is a P2X3 antagonist.

In one aspect is a method of treating pruritus in a mammal, the method comprising administering to the mammal a therapeutically effective amount of a P2X3 antagonist. In some embodiments is a method of treating pruritus in a mammal, comprising administering to the mammal a therapeutically effective amount of a P2X3 antagonist, wherein the P2X3 antagonist is a compound of Formula (I), or a pharmaceutically acceptable salt thereof:

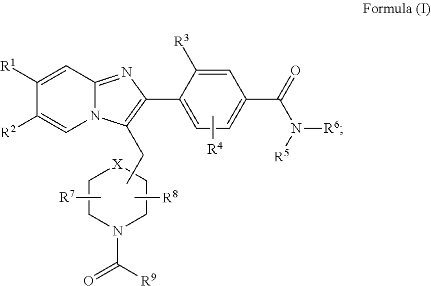

wherein:

R¹ is selected from the group consisting of cyano, halogen, methyl, and ethyl;

R² is selected from the group consisting of hydrogen, halogen, methyl, and ethyl;

R³ is selected from the group consisting of halogen, methyl, and ethyl;

R⁴ is selected from the group consisting of hydrogen, halogen, methyl, ethyl, and methoxy;

R⁵ and R⁶ are independently selected from the group consisting of hydrogen, C₁-C₆-alkyl, and hydroxy-C₁-C₆-alkyl; or
R⁵ and R⁶, together with the nitrogen to which they are both attached, form a 5- or 6-member heterocycloalkyl, wherein the heterocycloalkyl is optionally substituted with one or more substituents independently selected from the group consisting of halogen, hydroxyl, and C₁-C₄-alkyl;

R⁷ and R⁸ are independently selected from the group consisting of hydrogen and C₁-C₄-alkyl;

R⁹ is selected from the group consisting of C₁-C₆-alkyl, C₃-C₆-cycloalkyl, C₁-C₆-alkyl-C₃-C₆-cycloalkyl, halo-C₁-C₆-alkyl, C₁-C₆-alkoxy, halo-C₁-C₆-alkoxy, and C₁-C₆-alkoxy-C₁-C₆-alkyl; and
X is selected from the group consisting of a bond, CH₂, and O.

In some embodiments, R¹ is methyl. In some embodiments, R² is hydrogen. In some embodiments, R³ is fluoro. In some embodiments, X is O. In some embodiments, the compound of Formula (I) corresponds in structure to

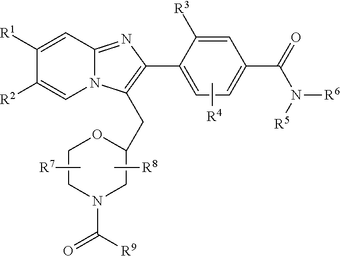

and R⁴ is selected from the group consisting of halogen, methyl, and ethyl. In some embodiments, R⁵ is hydrogen. In some embodiments, R⁶ is C₁-C₆-alkyl. In some embodiments, R⁶ is methyl. In some embodiments, R⁷ is hydrogen. In some embodiments, R⁸ is hydrogen. In some embodiments, R⁹ is C₁-C₆-alkoxy. In some embodiments, R⁹ is methoxy. In some embodiments, the compound of Formula (I) corresponds in structure to

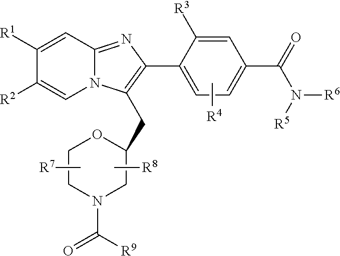

In some embodiments, the compound of Formula (I) corresponds in structure to:

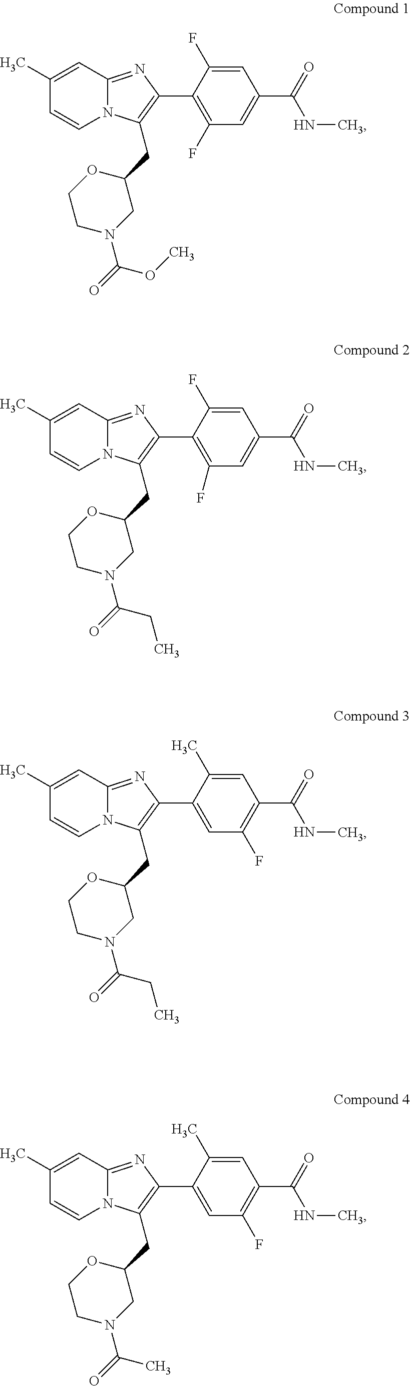 [SMILES: CNC(=O)c1cc(F)c(-c2nc3cc(C)ccn3c2C[C@H]2CN(C(=O)OC)CCO2)c(F)c1 ; CCC(=O)N1CCO[C@@H](Cc2c(-c3c(F)cc(C(=O)NC)cc3F)nc3cc(C)ccn23)C1 ; CCC(=O)N1CCO[C@@H](Cc2c(-c3cc(F)c(C(=O)NC)cc3C)nc3cc(C)ccn23)C1 ; CNC(=O)c1cc(C)c(-c2nc3cc(C)ccn3c2C[C@H]2CN(C(C)=O)CCO2)cc1F]

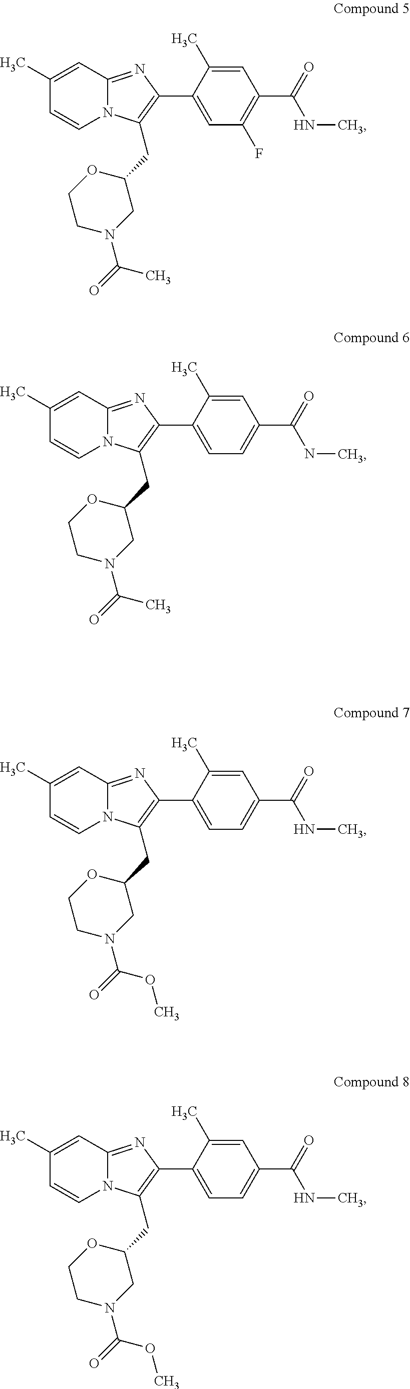 [SMILES: CNC(=O)c1cc(C)c(-c2nc3cc(C)ccn3c2C[C@@H]2CN(C(C)=O)CCO2)cc1F ; CNC(=O)c1ccc(-c2nc3cc(C)ccn3c2C[C@H]2CN(C(C)=O)CCO2)c(C)c1 ; CNC(=O)c1ccc(-c2nc3cc(C)ccn3c2C[C@H]2CN(C(=O)OC)CCO2)c(C)c1 ; CNC(=O)c1ccc(-c2nc3cc(C)ccn3c2C[C@@H]2CN(C(=O)OC)CCO2)c(C)c1]

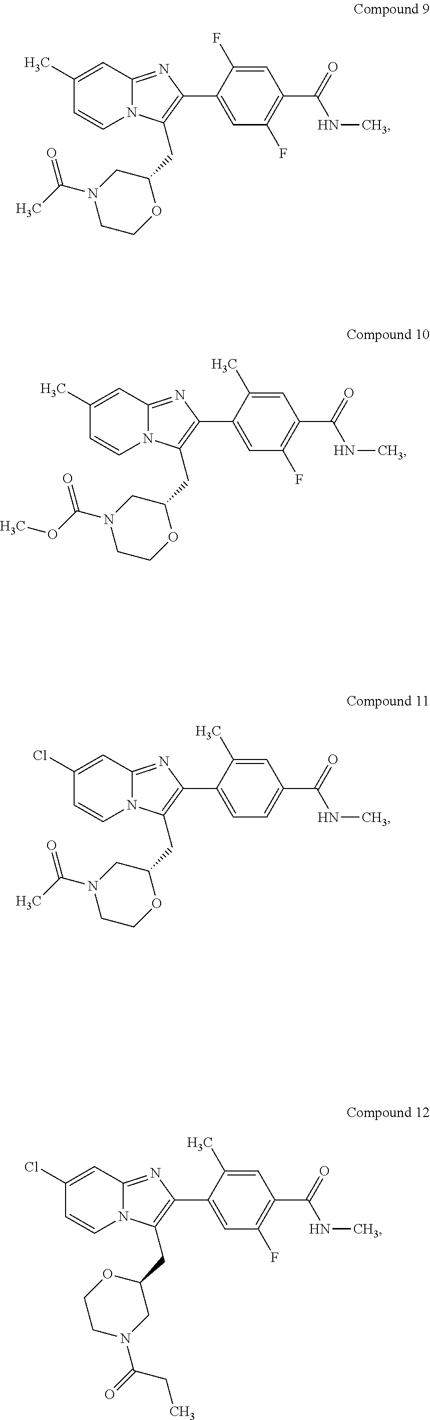 [SMILES: CNC(=O)c1cc(F)c(-c2nc3cc(C)ccn3c2C[C@H]2CN(C(C)=O)CCO2)cc1F ; CNC(=O)c1cc(C)c(-c2nc3cc(C)ccn3c2C[C@H]2CN(C(=O)OC)CCO2)cc1F ; CNC(=O)c1ccc(-c2nc3cc(Cl)ccn3c2C[C@H]2CN(C(C)=O)CCO2)c(C)c1 ; CCC(=O)N1CCO[C@@H](Cc2c(-c3cc(F)c(C(=O)NC)cc3C)nc3cc(Cl)ccn23)C1]

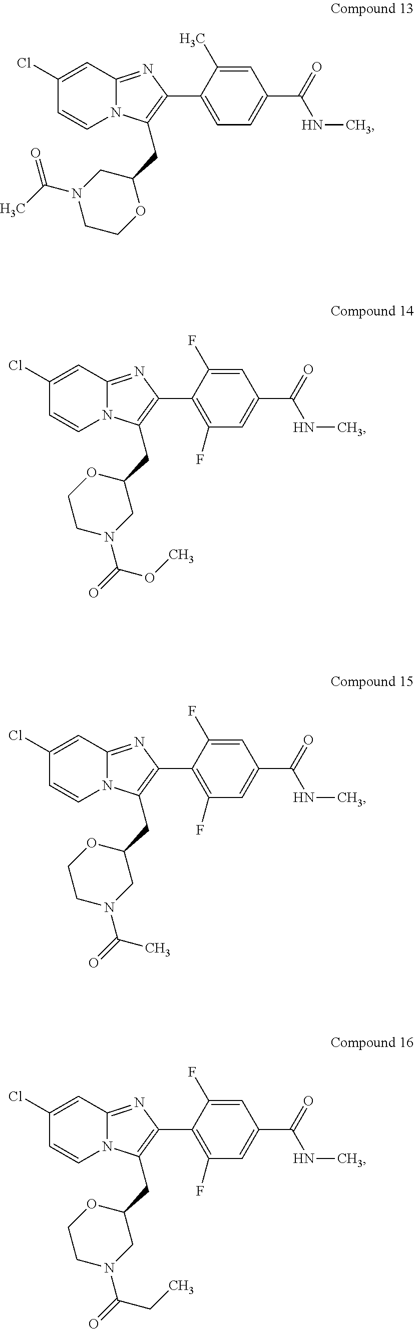 [SMILES: CNC(=O)c1ccc(-c2nc3cc(Cl)ccn3c2C[C@@H]2CN(C(C)=O)CCO2)c(C)c1 ; CNC(=O)c1cc(F)c(-c2nc3cc(Cl)ccn3c2C[C@H]2CN(C(=O)OC)CCO2)c(F)c1 ; CNC(=O)c1cc(F)c(-c2nc3cc(Cl)ccn3c2C[C@H]2CN(C(C)=O)CCO2)c(F)c1 ; CCC(=O)N1CCO[C@@H](Cc2c(-c3c(F)cc(C(=O)NC)cc3F)nc3cc(Cl)ccn23)C1]

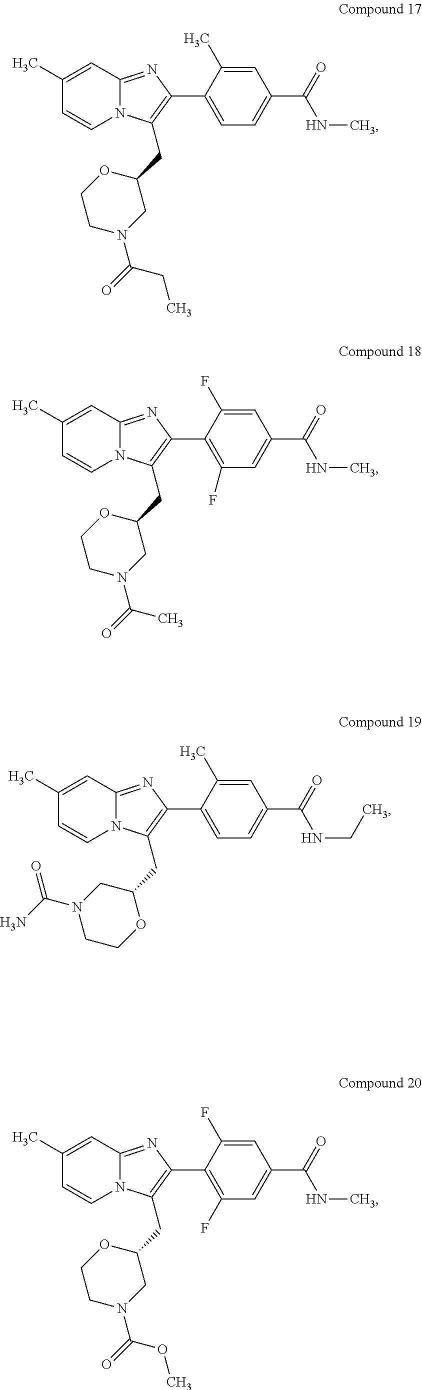 [SMILES: CCC(=O)N1CCO[C@@H](Cc2c(-c3ccc(C(=O)NC)cc3C)nc3cc(C)ccn23)C1 ; CNC(=O)c1cc(F)c(-c2nc3cc(C)ccn3c2C[C@H]2CN(C(C)=O)CCO2)c(F)c1 ; CCNC(=O)c1ccc(-c2nc3cc(C)ccn3c2C[C@H]2CN(C(N)=O)CCO2)c(C)c1 ; CNC(=O)c1cc(F)c(-c2nc3cc(C)ccn3c2C[C@@H]2CN(C(=O)OC)CCO2)c(F)c1]

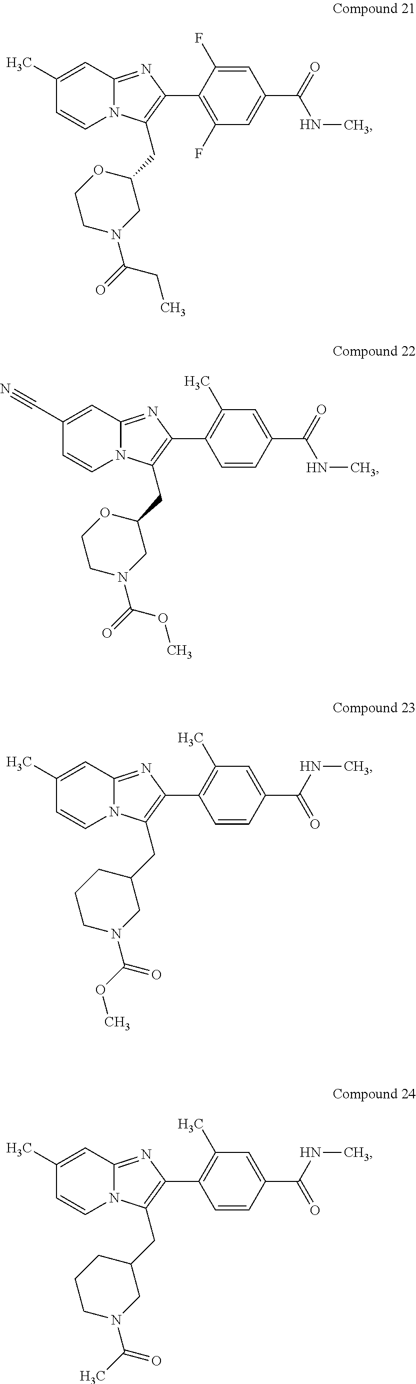 [SMILES: CCC(=O)N1CCO[C@H](Cc2c(-c3c(F)cc(C(=O)NC)cc3F)nc3cc(C)ccn23)C1 ; CNC(=O)c1ccc(-c2nc3cc(C#N)ccn3c2C[C@H]2CN(C(=O)OC)CCO2)c(C)c1 ; CNC(=O)c1ccc(-c2nc3cc(C)ccn3c2CC2CCCN(C(=O)OC)C2)c(C)c1 ; CNC(=O)c1ccc(-c2nc3cc(C)ccn3c2CC2CCCN(C(C)=O)C2)c(C)c1]

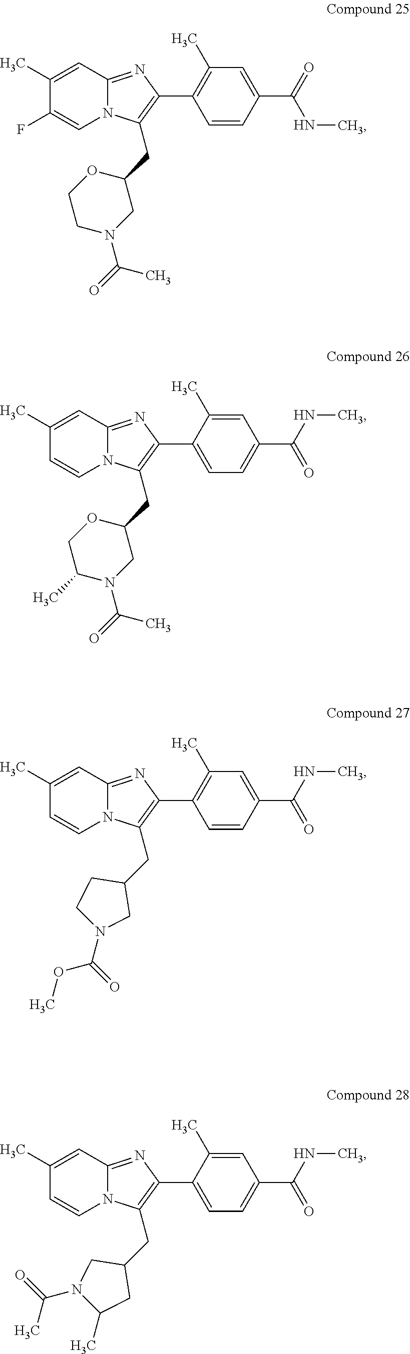 [SMILES: CNC(=O)c1ccc(-c2nc3cc(C)c(F)cn3c2C[C@H]2CN(C(C)=O)CCO2)c(C)c1 ; CNC(=O)c1ccc(-c2nc3cc(C)ccn3c2C[C@H]2CN(C(C)=O)[C@H](C)CO2)c(C)c1 ; CNC(=O)c1ccc(-c2nc3cc(C)ccn3c2CC2CCN(C(=O)OC)C2)c(C)c1 ; CNC(=O)c1ccc(-c2nc3cc(C)ccn3c2CC2CC(C)N(C(C)=O)C2)c(C)c1]

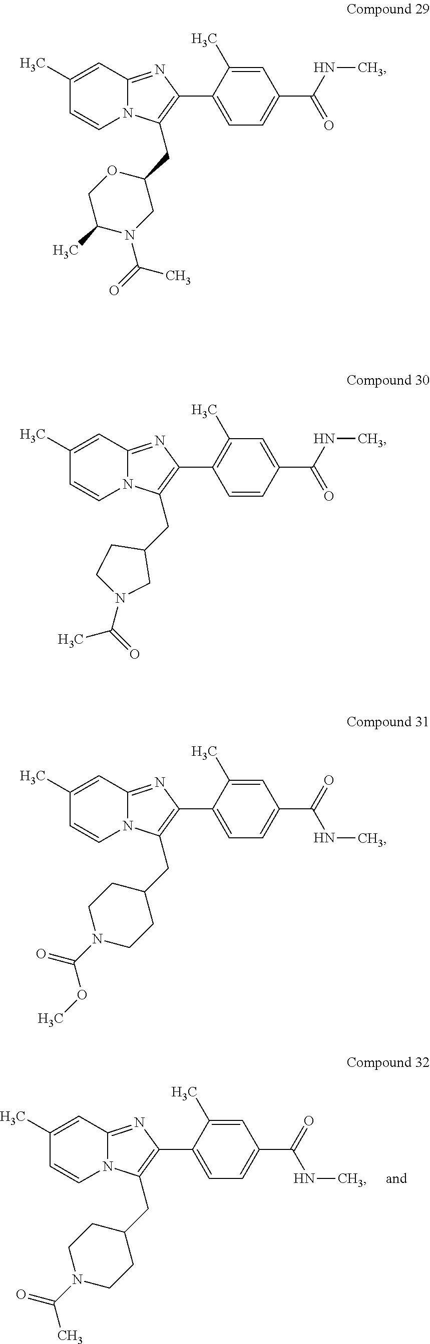 [SMILES: CNC(=O)c1ccc(-c2nc3cc(C)ccn3c2C[C@H]2CN(C(C)=O)[C@@H](C)CO2)c(C)c1 ; CNC(=O)c1ccc(-c2nc3cc(C)ccn3c2CC2CCN(C(C)=O)C2)c(C)c1 ; CNC(=O)c1ccc(-c2nc3cc(C)ccn3c2CC2CCN(C(=O)OC)CC2)c(C)c1 ; CNC(=O)c1ccc(-c2nc3cc(C)ccn3c2CC2CCN(C(C)=O)CC2)c(C)c1]

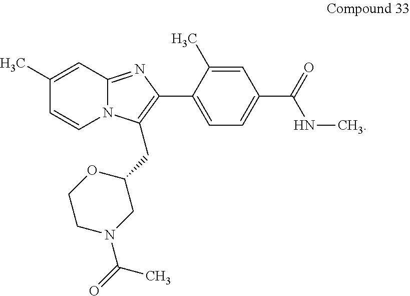 [SMILES: CNC(=O)c1ccc(-c2nc3cc(C)ccn3c2C[C@@H]2CN(C(C)=O)CCO2)c(C)c1]

In some embodiments, the compound of Formula (I) corresponds in structure to

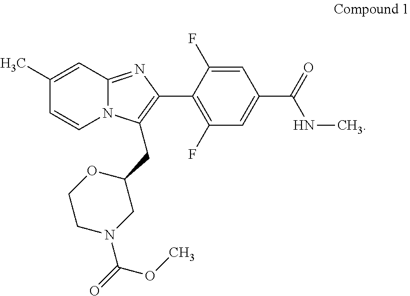 [SMILES: CNC(=O)c1cc(F)c(-c2nc3cc(C)ccn3c2C[C@H]2CN(C(=O)OC)CCO2)c(F)c1]

In some embodiments, the compound of Formula (I) corresponds in structure to

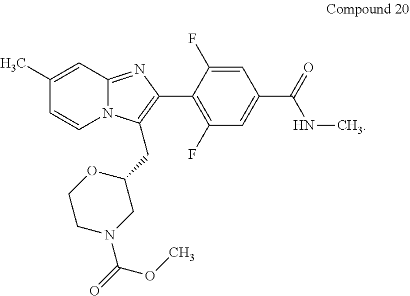 [SMILES: CNC(=O)c1cc(F)c(-c2nc3cc(C)ccn3c2C[C@@H]2CN(C(=O)OC)CCO2)c(F)c1]

In some embodiments, the compound of Formula (I) corresponds in structure to

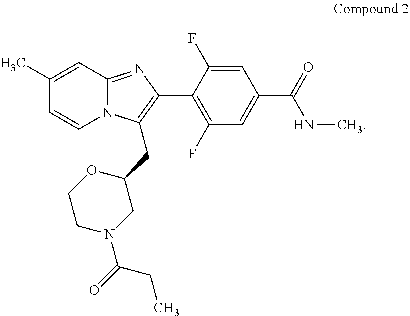 [SMILES: CCC(=O)N1CCO[C@@H](Cc2c(-c3c(F)cc(C(=O)NC)cc3F)nc3cc(C)ccn23)C1]

In some embodiments, the compound of Formula (I) corresponds in structure to

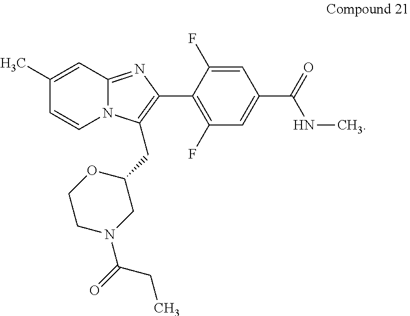 [SMILES: CCC(=O)N1CCO[C@H](Cc2c(-c3c(F)cc(C(=O)NC)cc3F)nc3cc(C)ccn23)C1]

In some embodiments, the P2X3 antagonist corresponds in structure to

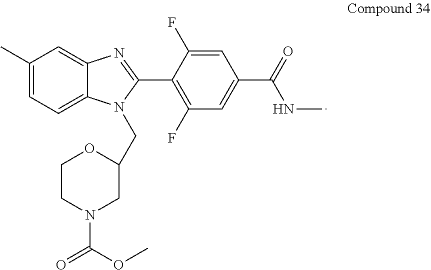

In some embodiments, the P2X3 antagonist corresponds in structure to

In some embodiments, the P2X3 antagonist corresponds in structure to

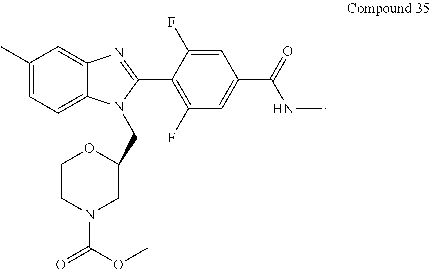

In some embodiments, the P2X3 antagonist corresponds in structure to

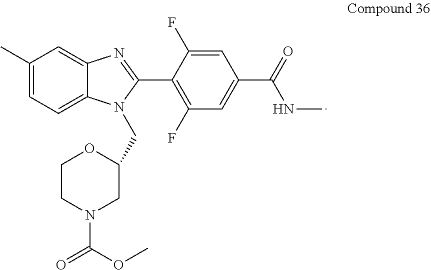

In some embodiments is a method of treating pruritus in a mammal, the method comprising administering to the mammal a therapeutically effective amount of a P2X3 antagonist wherein the mammal is human. In some embodiments is a method of treating pruritus in a mammal wherein the pruritus is associated with an inflammatory skin disease, an infectious skin disease, an autoimmune skin disease, or a pregnancy-related skin disease. In some embodiments is a method of treating pruritus in a mammal wherein the pruritus is associated with an inflammatory skin disease selected from the group consisting of atopic dermatitis, allergic, irritant contact dermatitis, exsiccation dermatitis, nummular and dyshidrotic dermatitis, lichen planus, lichen sclerosus et atrophicus, polymorphous light eruption psoriasis, Grover's disease, mucinosis, mastocytosis, and urticaria. In some embodiments is a method of treating pruritus in a mammal wherein the pruritus is associated with an infectious skin disease selected from the group consisting of mycoses, bacterial and viral infections, scabies, pediculosis, insect bites, and folliculitides. In some embodiments is a method of treating pruritus in a mammal wherein the pruritus is associated with an autoimmune skin disease selected from the group consisting of dermatitis herpetiformis (Duhring's disease), bullous pemphigoid; genodermatoses, Darier's disease, and Hailey-Hailey disease. In some embodiments is a method of treating pruritus in a mammal wherein the pruritus is associated with a pregnancy-related skin disease selected from the group consisting of polymorphic eruption of pregnancy (PEP), atopic eruption of pregnancy, pemphigoid gestationis, neoplasias, and cutaneous T-cell lymphoma. In some embodiments is a method of treating pruritus in a mammal wherein the pruritus is associated with a kidney disease or a therapeutic procedure to treat a kidney disease. In some embodiments is a method of treating pruritus in a mammal wherein the pruritus is associated with a chronic kidney disease. In some embodiments is a method of treating pruritus in a mammal wherein the pruritus is associated with a therapeutic procedure to treat a kidney disease, wherein the therapeutic procedure to treat the kidney disease is hemodialysis or peritoneal dialysis. In some embodiments is a method of treating pruritus in a mammal wherein the pruritus is associated with a medical procedure or treatment. In some embodiments is a method of treating pruritus in a mammal wherein the pruritus is associated with a medical treatment with a drug selected from the group consisting of opioids, anti-malarial drugs, anti-cancer therapies, and epidermal growth factor receptor inhibitors. In some embodiments is a method of treating pruritus in a mammal wherein the pruritus is associated with prurigo nodularis. In some embodiments is a method of treating pruritus in a mammal wherein the P2X3 antagonist is formulated for administration to a mammal by intravenous administration, subcutaneous administration, oral administration, inhalation, nasal administration, topical administration, or ophthalmic administration. In some embodiments is a method of treating pruritus in a mammal wherein the P2X3 antagonist is formulated in the form of a tablet, a pill, a capsule, a liquid, a suspension, a gel, a dispersion, a solution, an emulsion, an ointment, or a lotion. In some embodiments is a method of treating pruritus in a mammal, the method comprising administering to the mammal a therapeutically effective amount of a P2X3 antagonist, further comprising the administration of a second therapeutic agent. In some embodiments is a method of treating pruritus in a mammal, the method comprising administering to the mammal a therapeutically effective amount of a P2X3 antagonist, further comprising the administration of a NK-1 antagonist. In some embodiments is a method of treating pruritus in a mammal, the method comprising administering to the mammal a therapeutically effective amount of a P2X3 antagonist, further comprising the administration of a NK-1 antagonist wherein the NK-1 antagonist is selected from the group consisting of serlopitant, orvepitant, rolapitant, aprepitant, and fosaprepitant, or a pharmaceutically acceptable salt thereof. In some embodiments is a method of treating pruritus in a mammal, the method comprising administering to the mammal a therapeutically effective amount of a P2X3 antagonist, further comprising the administration of a NK-1 antagonist wherein the NK-1 antagonist is selected from the group consisting of serlopitant, aprepitant, casopitant, dapitant, ezlopitant, fosaprepitant, lanepitant, maropitant, netupitant, nolpitant, orvepitant, rolapitant, vestipitant, vofopitant, AV-818, BIIF 1149CL, CP122,721, DNK-333, GSK-424887, L-733060, L-759274, LY-686017, M516102, and TA-5538.

### BRIEF DESCRIPTION OF THE DRAWINGS

FIG. 1 depicts the effect of 10 μM or 50 μM α,β-methylene-adenosine 5′-triphosphate (α,β-Me-ATP) on low dose chloroquine (CQ) induced itch behavior.

FIG. 2 shows the effect of Compound 1 (three separate doses) and U50,488 on low dose chloroquine CQ-induced plus 50 μM α,β-Me-ATP itch behavior as measured by number of scratches induced in 15 minutes post dose administration.

FIG. 3 shows the effect of Compound 1 (10 mpk) on low dose chloroquine CQ-induced plus 100 μM α,β-Me-ATP itch behavior as measured by number of scratches induced in 15 minutes post dose administration.

FIG. 4 shows the effect of Compound 1 (10 mpk) on high dose chloroquine CQ-induced itch behavior as measured by number of scratches induced in 30 minutes post dose administration.

FIG. 5 shows the effect of Compound 1 (2, 10, and 50 mg/kg) and U50,488 (3 mg/kg) on chronic itch behavior as measured by the number of spontaneous scratches in 60 minutes on Day 10 in the AEW (acetone-ether-water) dry skin model.

FIG. 6 shows the effect of Compound 1 (2, 10, and 50 mg/kg) and U50,488 (3 mg/kg) on chronic itch behavior as measured by the number of spontaneous scratches in 10 minute intervals on Day 10 in the AEW (acetone-ether-water) dry skin model.

FIG. 7 shows the effect of Compound 1 (2, 10, and 50 mg/kg) and U50,488 (3 mg/kg) on chronic itch behavior as measured by the number of spontaneous scratches in 60 minutes on Day 8 in the MC903 atopic dermatitis model.

FIG. 8 shows the effect of Compound 1 (2, 10, and 50 mg/kg) and U50,488 (3 mg/kg) on chronic itch behavior as measured by the number of spontaneous scratches in 10 minute intervals on Day 8 in the MC903 atopic dermatitis model.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Pruritogenic stimuli can be induced by mechanical, thermal and chemical means, which are sensed by afferent neurons innervating the skin and transmitted to the thalamus for processing and reflex initiation. Stimuli and afferent transmission acts through a wide variety of afferent neurons (pruriceptive neurons), which are a population partially overlapping in molecular phenotype with pain-sensing neurons in the skin. Pruriceptive neurons can respond to a wide variety of stimuli, but pathological itch is induced primarily by endogenous chemical agents (e.g. histamine, substance P, gastrin-release peptide, interleukins, nerve growth factors) acting at neuron terminals in the skin. These pruritogenic agents are released in the context of disorders with excessive inflammation (e.g. atopic dermatitis, psoriasis), systemic disease (e.g. chronic liver and kidney disease) neuropathic disorders (e.g. post-herpetic itch), or psychogenic conditions (e.g. obsessive compulsive disorder, substance abuse) (Yosipovitch et al., N. Engl. J. Med., 2013, 1625-1634).

Pruriceptive afferent neurons are characterized as c- or aδ-fibers of the dorsal root ganglions that innervate skin tissues and form synapses with the spinal cord. C- and aδ-fibers terminals in the skin express receptors responding to pruritogenic chemical agents to initiate action potentials that are transmitted to the CNS. These neurons also express P2X3 cation channels that regulate neuronal sensitivity to excitation by a pruritogenic stimuli. Notably, P2X3 channels are co-expressed on the cell membrane of MgprA3+ neurons, the major pruriceptive neuron phenotype innervating the skin, and the number of these neurons is increased in mouse models of chronic itch (Han et al., Nat. Neurosci., 2013, 174-182; Zhao et al., J. Clin. Invest., 2013, 4769-4780).

P2X3 channels are neuronal excitability regulators that are activated by local release of ATP, a neurotransmitter and extracellular messenger with pro-inflammatory properties. ATP is well established as an important chemical messenger released in excess by neuronal and non-neuronal cell types in multiple pathological conditions (Burnstock, Front. Pharmacol., 2017, 661; Burnstock, Biochem. Pharmacol., 2017, doi:10.1016/j.bcp.2017.07.016). Accordingly, the increased release of ATP can lead to hyperexcitability of afferent pruriceptive neurons and heightened sensitivity to any pruritogenic agent released pathologically in the skin. Overall, P2X3 channels acting through pathological ATP release may be potentially relevant targets to modulate the sensitivity of afferent neurons to itch sensations. Their inhibition could offer an approach to dampen peripheral hypersensitivity to itch in various diseases, with a broad mechanism independent of the pathological stimuli acting at itch receptors.

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, or process, or the like, described herein, may “consist of” or “consist essentially of” the described features.

As used in the specification and appended claims, unless specified to the contrary, the following terms have the meaning indicated below.

As used herein, C₁-Cₓ includes C₁-C₂, C₁-C₃ . . . C₁-Cₓ. C₁-Cₓ refers to the number of carbon atoms that make up the moiety to which it designates (excluding optional substituents).

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” or “alkylene” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to six carbon atoms (e.g., C₁-C₆ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₇ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), and 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, aryl, aralkyl, heterocycloalkyl, heteroaryl or heteroarylalkyl, and each R is independently alkyl, fluoroalkyl, cycloalkyl, aryl, aralkyl, heterocycloalkyl, heteroaryl or heteroarylalkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{f}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, aryl, aralkyl, heterocycloalkyl, heteroaryl or heteroarylalkyl, and each R is independently alkyl, fluoroalkyl, cycloalkyl, aryl, aralkyl, heterocycloalkyl, heteroaryl or heteroarylalkyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, aryl, aralkyl, heterocycloalkyl, heteroaryl or heteroarylalkyl, and each R is independently alkyl, fluoroalkyl, cycloalkyl, aryl, aralkyl, heterocycloalkyl, heteroaryl or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C (O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“Aryloxy” refers to a radical bonded through an oxygen atom of the formula —O-aryl, where aryl is as defined above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkyloxy” refers to a radical bonded through an oxygen atom of the formula —O— aralkyl, where aralkyl is as defined above.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynyl ene chain as defined above. The aryl part of the aralkynyl radical is optionally substituted as described above for an aryl group. The alkynyl ene chain part of the aralkynyl radical is optionally substituted as defined above for an alkynylene chain.

“Cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. The cycloalkyl is attached to the rest of the molecule by a single bond. Cycloalkyls are saturated, (i.e., containing single C—C bonds only) or partially unsaturated (i.e., containing one or more double bonds or triple bonds.) Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). A partially unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “cycloalkyl” is meant to include cycloalkyl radicals optionally substituted by one or more substituents independently selected from alkyl, alkenyl, alkynyl, halo, fluoroalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C (O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“Halo” or “halogen” refers to bromo, chloro, fluoro or iodo substituents.

“Haloalkyl” refers to an alkyl radical, as defined above, that is substituted by one or more halo radicals, as defined above.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, l-fluoromethyl-2-fluoroethyl, and the like. The alkyl part of the fluoroalkyl radical are optionally substituted as defined above for an alkyl group.

“Haloalkoxy” refers to an alkoxy radical, as defined above, that is substituted by one or more halo radicals, as defined above.

“Heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocycloalkyl radical is a monocyclic, bicyclic, tricyclic or tetracyclic ring system, which include fused, spiro, or bridged ring systems. The heteroatoms in the heterocycloalkyl radical are optionally oxidized. One or more nitrogen atoms, if present, are optionally quaternized. The heterocycloalkyl radical is partially or fully saturated. In some embodiments, the heterocycloalkyl is attached to the rest of the molecule through any atom of the ring(s).

Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C (O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“Heteroaryl” refers to a radical derived from a 5- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“N-heteroaryl” refers to a heteroaryl radical as defined above containing at least one nitrogen and where the point of attachment of the heteroaryl radical to the rest of the molecule is through a nitrogen atom in the heteroaryl radical. An A-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O— heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, he compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans) Likewise, all possible isomers, as well as their racemic and optically pure forms, and all tautomeric forms are also intended to be included. The term “geometric isomer” refers to E or Z geometric isomers (e.g., cis or tram) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

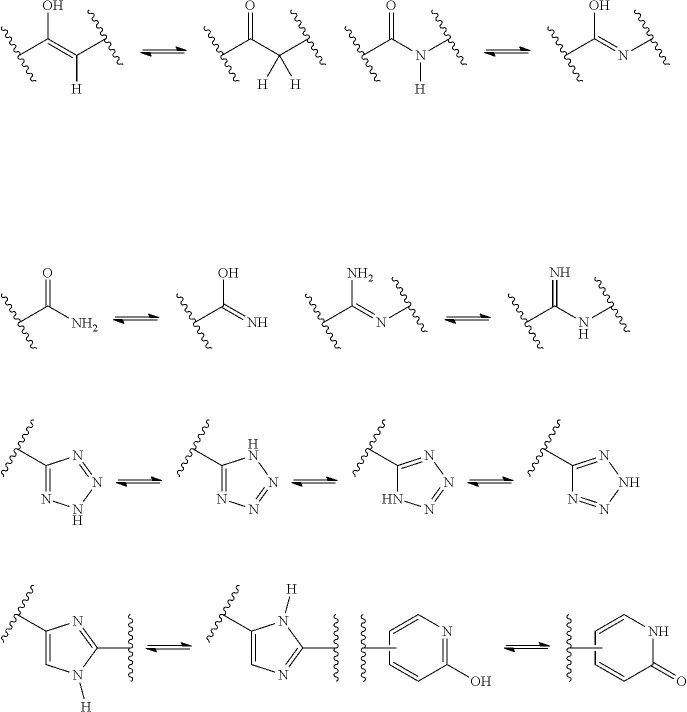

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Prodrugs”, includes compounds that, after administration, are metabolized into a pharmacologically active drug (R. B. Silverman, 1992, “The Organic Chemistry of Drug Design and Drug Action,” Academic Press, Chp. 8). A prodrug may be used to improve how a compound is absorbed, distributed, metabolized, and excreted.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et ah, “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Methods

In some embodiments is a method of treating pruritus in a mammal in need thereof, the method comprising administering to the mammal a therapeutically effective amount of a P2X3 antagonist. In some embodiments is a method of treating pruritus in a mammal in need thereof, the method comprising administering to the mammal a therapeutically effective amount of a P2X3 antagonist, wherein the P2X3 antagonist is a compound of Formula (I), or a pharmaceutically acceptable salt thereof, having the structure:

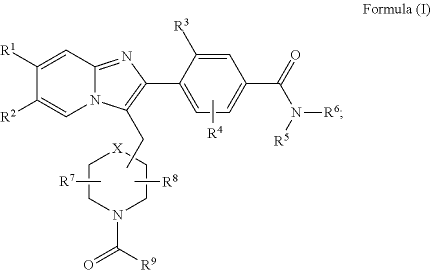

wherein:
R¹ is selected from the group consisting of cyano, halogen, methyl, and ethyl;

R² is selected from the group consisting of hydrogen, halogen, methyl, and ethyl;

R³ is selected from the group consisting of halogen, methyl, and ethyl;

R⁴ is selected from the group consisting of hydrogen, halogen, methyl, ethyl, and methoxy;

R⁵ and R⁶ are independently selected from the group consisting of hydrogen, C₁-C₆-alkyl, and hydroxy-C₁-C₆-alkyl; or
R⁵ and R⁶, together with the nitrogen to which they are both attached, form a 5- or 6-member heterocycloalkyl, wherein the heterocycloalkyl is optionally substituted with one or more substituents independently selected from the group consisting of halogen, hydroxyl, and C₁-C₄-alkyl;

R⁷ and R⁸ are independently selected from the group consisting of hydrogen and C₁-C₄-alkyl;

R⁹ is selected from the group consisting of C₁-C₆-alkyl, C₃-C₆-cycloalkyl, C₁-C₆-alkyl-C₃-C₆-cycloalkyl, halo-C₁-C₆-alkyl, C₁-C₆-alkoxy, halo-C₁-C₆-alkoxy, and C₁-C₆-alkoxy-C₁-C₆-alkyl; and
X is selected from the group consisting of a bond, CH₂, and O.

In some embodiments of the methods described herein, the P2X3 antagonist is a compound of Formula (I), or a pharmaceutically acceptable salt thereof, wherein X is a bond. In some embodiments of the methods described herein, the P2X3 antagonist is a compound of Formula (I), or a pharmaceutically acceptable salt thereof, wherein X is CH₂. In some embodiments of the methods described herein, the P2X3 antagonist is a compound of Formula (I), or a pharmaceutically acceptable salt thereof, wherein X is O.

In some embodiments of the methods described herein, the P2X3 antagonist is a compound of Formula (I), or a pharmaceutically acceptable salt thereof, wherein R¹ is cyano. In some embodiments of the methods described herein, the P2X3 antagonist is a compound of Formula (I), or a pharmaceutically acceptable salt thereof, wherein R¹ is halogen. In some embodiments of the methods described herein, the P2X3 antagonist is a compound of Formula (I), or a pharmaceutically acceptable salt thereof, wherein R¹ is methyl. In some embodiments of the methods described herein, the P2X3 antagonist is a compound of Formula (I), or a pharmaceutically acceptable salt thereof, wherein R¹ is ethyl.

In some embodiments of the methods described herein, the P2X3 antagonist is a compound of Formula (I), or a pharmaceutically acceptable salt thereof, wherein R² is hydrogen. In some embodiments of the methods described herein, the P2X3 antagonist is a compound of Formula (I), or a pharmaceutically acceptable salt thereof, wherein R² is halogen. In some embodiments of the methods described herein, the P2X3 antagonist is a compound of Formula (I), or a pharmaceutically acceptable salt thereof, wherein R² is methyl. In some embodiments of the methods described herein, the P2X3 antagonist is a compound of Formula (I), or a pharmaceutically acceptable salt thereof, wherein R² is ethyl.

In some embodiments of the methods described herein, the P2X3 antagonist is a compound of Formula (I), or a pharmaceutically acceptable salt thereof, wherein R³ is halogen. In some embodiments of the methods described herein, the P2X3 antagonist is a compound of Formula (I), or a pharmaceutically acceptable salt thereof, wherein R³ is fluoro. In some embodiments of the methods described herein, the P2X3 antagonist is a compound of Formula (I), or a pharmaceutically acceptable salt thereof, wherein R³ is methyl. In some embodiments of the methods described herein, the P2X3 antagonist is a compound of Formula (I), or a pharmaceutically acceptable salt thereof, wherein R³ is ethyl.

In some embodiments of the methods described herein, the P2X3 antagonist is a compound of Formula (I), or a pharmaceutically acceptable salt thereof, wherein R⁴ is hydrogen. In some embodiments of the methods described herein, the P2X3 antagonist is a compound of Formula (I), or a pharmaceutically acceptable salt thereof, wherein R⁴ is halogen. In some embodiments of the methods described herein, the P2X3 antagonist is a compound of Formula (I), or a pharmaceutically acceptable salt thereof, wherein R⁴ is fluoro. In some embodiments of the methods described herein, the P2X3 antagonist is a compound of Formula (I), or a pharmaceutically acceptable salt thereof, wherein R⁴ is methyl. In some embodiments of the methods described herein, the P2X3 antagonist is a compound of Formula (I), or a pharmaceutically acceptable salt thereof, wherein R⁴ is ethyl. In some embodiments of the methods described herein, the P2X3 antagonist is a compound of Formula (I), or a pharmaceutically acceptable salt thereof, wherein R⁴ is methoxy.

In some embodiments of the methods described herein, the P2X3 antagonist is a compound of Formula (I), or a pharmaceutically acceptable salt thereof, wherein R⁵ and R⁶ are independently selected from the group consisting of hydrogen and C₁-C₆-alkyl. In some embodiments of the methods described herein, the P2X3 antagonist is a compound of Formula (I), or a pharmaceutically acceptable salt thereof, wherein R⁵ and R⁶ are each hydrogen. In some embodiments of the methods described herein, the P2X3 antagonist is a compound of Formula (I), or a pharmaceutically acceptable salt thereof, wherein R⁵ and R⁶ are each C₁-C₆-alkyl. In some embodiments of the methods described herein, the P2X3 antagonist is a compound of Formula (I), or a pharmaceutically acceptable salt thereof, wherein R⁵ is hydrogen and R⁶ is C₁-C₆-alkyl. In some embodiments of the methods described herein, the P2X3 antagonist is a compound of Formula (I), or a pharmaceutically acceptable salt thereof, wherein R⁵ is hydrogen and R⁶ is methyl.

In some embodiments of the methods described herein, the P2X3 antagonist is a compound of Formula (I), or a pharmaceutically acceptable salt thereof, wherein R⁷ and R⁸ are independently selected from the group consisting of hydrogen and methyl. In some embodiments of the methods described herein, the P2X3 antagonist is a compound of Formula (I), or a pharmaceutically acceptable salt thereof, wherein R⁷ and R⁸ are each hydrogen. In some embodiments of the methods described herein, the P2X3 antagonist is a compound of Formula (I), or a pharmaceutically acceptable salt thereof, wherein R⁷ is hydrogen and R⁸ is methyl.

In some embodiments of the methods described herein, the P2X3 antagonist is a compound of Formula (I), or a pharmaceutically acceptable salt thereof, wherein R⁹ is selected from the group consisting of C₁-C₆-alkyl and C₁-C₆-alkoxy. In some embodiments of the methods described herein, the P2X3 antagonist is a compound of Formula (I), or a pharmaceutically acceptable salt thereof, wherein R⁹ is C₁-C₆-alkyl. In some embodiments of the methods described herein, the P2X3 antagonist is a compound of Formula (I), or a pharmaceutically acceptable salt thereof, wherein R⁹ is methyl. In some embodiments of the methods described herein, the P2X3 antagonist is a compound of Formula (I), or a pharmaceutically acceptable salt thereof, wherein R⁹ is ethyl. In some embodiments of the methods described herein, the P2X3 antagonist is a compound of Formula (I), or a pharmaceutically acceptable salt thereof, wherein R⁹ is C₁-C₆-alkoxy. In some embodiments of the methods described herein, the P2X3 antagonist is a compound of Formula (I), or a pharmaceutically acceptable salt thereof, wherein R⁹ is methoxy.

In some embodiments of the methods described herein, the P2X3 antagonist is a compound of Formula (I), or a pharmaceutically acceptable salt thereof, wherein the compound of Formula (I) corresponds in structure to

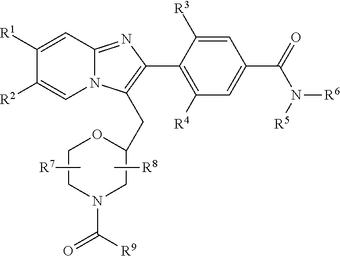

and R⁴ is selected from the group consisting of halogen, methyl, and ethyl. In some embodiments of the methods described herein, the P2X3 antagonist is a compound of Formula (I), or a pharmaceutically acceptable salt thereof, wherein the compound of Formula (I) corresponds in structure to

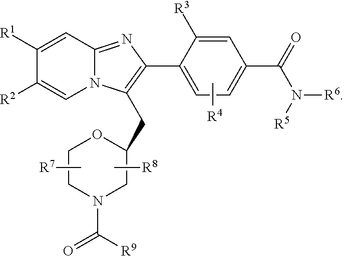

In some embodiments of the methods described herein, the P2X3 antagonist is a compound of Formula (I), or a pharmaceutically acceptable salt thereof, wherein X is O, R¹ is methyl, R² is hydrogen, R³ is halogen, R⁴ is halogen, R⁵ is hydrogen, R⁶ is C₁-C₆-alkyl, R⁷ is hydrogen, R⁸ is hydrogen, and R⁹ is C₁-C₆-alkyl. In some embodiments of the methods described herein, the P2X3 antagonist is a compound of Formula (I), or a pharmaceutically acceptable salt thereof, wherein X is O, R¹ is methyl, R² is hydrogen, R³ is fluoro, R⁴ is fluoro, R⁵ is hydrogen, R⁶ is methyl, R⁷ is hydrogen, R⁸ is hydrogen, and R⁹ is methyl.

In some embodiments of the methods described herein, the P2X3 antagonist is a compound of Formula (I), or a pharmaceutically acceptable salt thereof, wherein X is O, R¹ is methyl, R² is hydrogen, R³ is halogen, R⁴ is halogen, R⁵ is hydrogen, R⁶ is C₁-C₆-alkyl, R⁷ is hydrogen, R⁸ is hydrogen, and R⁹ is C₁-C₆-alkoxy. In some embodiments of the methods described herein, the P2X3 antagonist is a compound of Formula (I), or a pharmaceutically acceptable salt thereof, wherein X is O, R¹ is methyl, R² is hydrogen, R³ is fluoro, R⁴ is fluoro, R⁵ is hydrogen, R⁶ is methyl, R⁷ is hydrogen, R⁸ is hydrogen, and R⁹ is methoxy.

In some embodiments of the methods described herein, the P2X3 antagonist is a compound of Formula (I), or a pharmaceutically acceptable salt thereof, wherein X is O, R¹ is methyl, R² is hydrogen, R³ is methyl, R⁴ is hydrogen, R⁵ is hydrogen, R⁶ is C₁-C₆-alkyl, R⁷ is hydrogen, R is hydrogen, and R is C₁-C₆-alkyl. In some embodiments of the methods described herein, the P2X3 antagonist is a compound of Formula (I), or a pharmaceutically acceptable salt thereof, wherein X is O, R¹ is methyl, R² is hydrogen, R³ is methyl, R⁴ is hydrogen, R⁵ is hydrogen, R⁶ is methyl, R⁷ is hydrogen, R⁸ is hydrogen, and R⁹ is methyl.

In some embodiments of the methods described herein, the P2X3 antagonist is a compound of Formula (I), or a pharmaceutically acceptable salt thereof, wherein X is O, R¹ is methyl, R² is hydrogen, R³ is methyl, R⁴ is hydrogen, R⁵ is hydrogen, R⁶ is C₁-C₆-alkyl, R⁷ is hydrogen, R⁸ is hydrogen, and R⁹ is C₁-C₆-alkoxy. In some embodiments of the methods described herein, the P2X3 antagonist is a compound of Formula (I), or a pharmaceutically acceptable salt thereof, wherein X is O, R¹ is methyl, R² is hydrogen, R³ is methyl, R⁴ is hydrogen, R⁵ is hydrogen, R⁶ is methyl, R⁷ is hydrogen, R⁸ is hydrogen, and R⁹ is methoxy.

In some embodiments of the methods described herein, the P2X3 antagonist is a compound of Formula (I), or a pharmaceutically acceptable salt thereof, wherein the compound of Formula (I) corresponds in structure to:

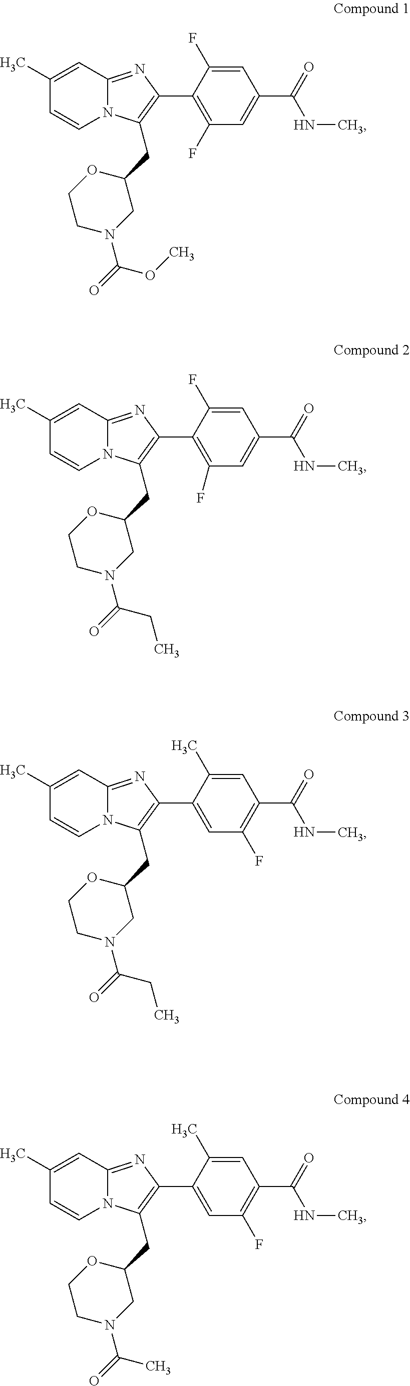 [SMILES: CNC(=O)c1cc(F)c(-c2nc3cc(C)ccn3c2C[C@H]2CN(C(=O)OC)CCO2)c(F)c1 ; CCC(=O)N1CCO[C@@H](Cc2c(-c3c(F)cc(C(=O)NC)cc3F)nc3cc(C)ccn23)C1 ; CCC(=O)N1CCO[C@@H](Cc2c(-c3cc(F)c(C(=O)NC)cc3C)nc3cc(C)ccn23)C1 ; CNC(=O)c1cc(C)c(-c2nc3cc(C)ccn3c2C[C@H]2CN(C(C)=O)CCO2)cc1F]

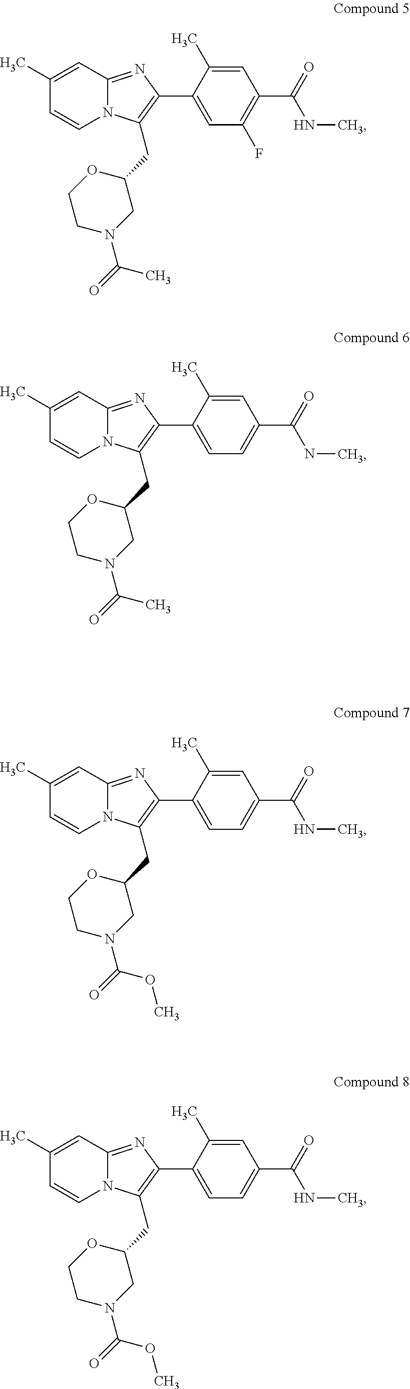 [SMILES: CNC(=O)c1cc(C)c(-c2nc3cc(C)ccn3c2C[C@@H]2CN(C(C)=O)CCO2)cc1F ; CNC(=O)c1ccc(-c2nc3cc(C)ccn3c2C[C@H]2CN(C(C)=O)CCO2)c(C)c1 ; CNC(=O)c1ccc(-c2nc3cc(C)ccn3c2C[C@H]2CN(C(=O)OC)CCO2)c(C)c1 ; CNC(=O)c1ccc(-c2nc3cc(C)ccn3c2C[C@@H]2CN(C(=O)OC)CCO2)c(C)c1]

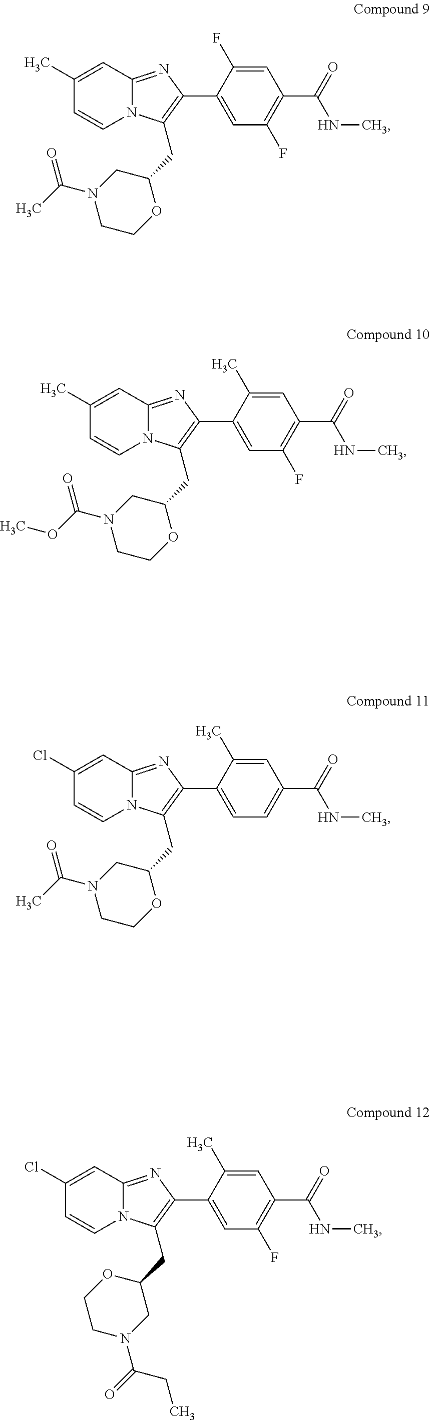 [SMILES: CNC(=O)c1cc(F)c(-c2nc3cc(C)ccn3c2C[C@H]2CN(C(C)=O)CCO2)cc1F ; CNC(=O)c1cc(C)c(-c2nc3cc(C)ccn3c2C[C@H]2CN(C(=O)OC)CCO2)cc1F ; CNC(=O)c1ccc(-c2nc3cc(Cl)ccn3c2C[C@H]2CN(C(C)=O)CCO2)c(C)c1 ; CCC(=O)N1CCO[C@@H](Cc2c(-c3cc(F)c(C(=O)NC)cc3C)nc3cc(Cl)ccn23)C1]

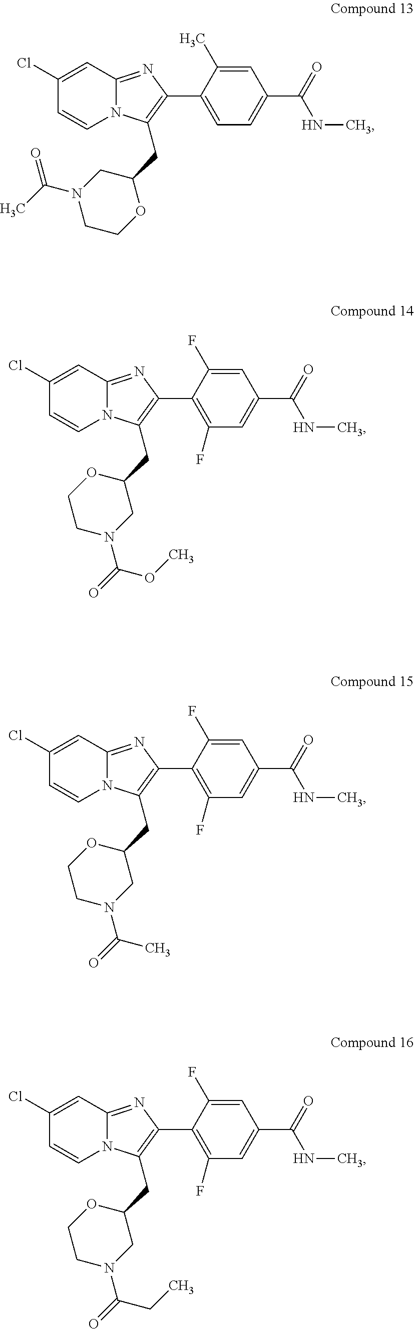 [SMILES: CNC(=O)c1ccc(-c2nc3cc(Cl)ccn3c2C[C@@H]2CN(C(C)=O)CCO2)c(C)c1 ; CNC(=O)c1cc(F)c(-c2nc3cc(Cl)ccn3c2C[C@H]2CN(C(=O)OC)CCO2)c(F)c1 ; CNC(=O)c1cc(F)c(-c2nc3cc(Cl)ccn3c2C[C@H]2CN(C(C)=O)CCO2)c(F)c1 ; CCC(=O)N1CCO[C@@H](Cc2c(-c3c(F)cc(C(=O)NC)cc3F)nc3cc(Cl)ccn23)C1]

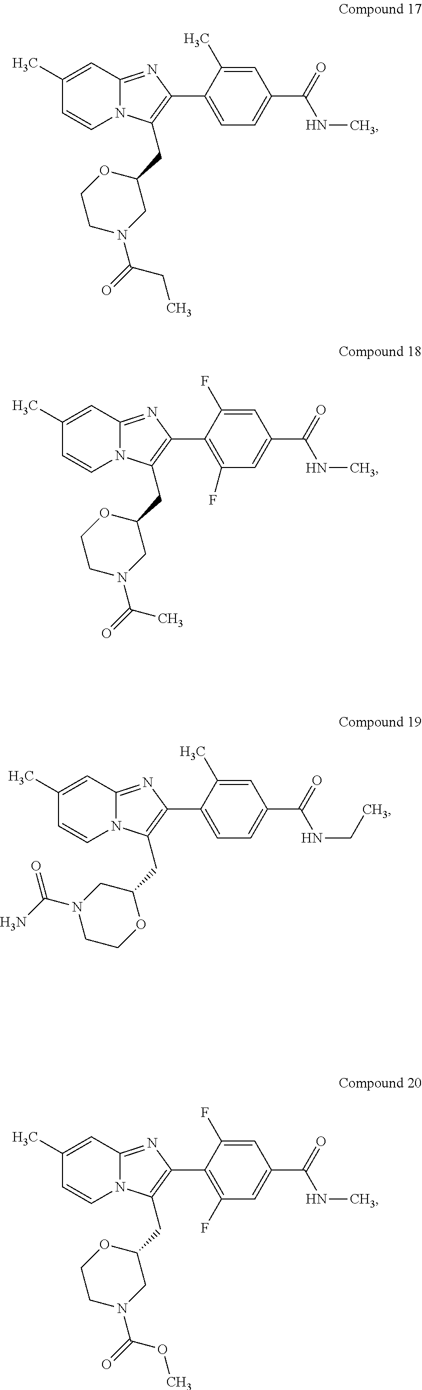 [SMILES: CCC(=O)N1CCO[C@@H](Cc2c(-c3ccc(C(=O)NC)cc3C)nc3cc(C)ccn23)C1 ; CNC(=O)c1cc(F)c(-c2nc3cc(C)ccn3c2C[C@H]2CN(C(C)=O)CCO2)c(F)c1 ; CCNC(=O)c1ccc(-c2nc3cc(C)ccn3c2C[C@H]2CN(C(N)=O)CCO2)c(C)c1 ; CNC(=O)c1cc(F)c(-c2nc3cc(C)ccn3c2C[C@@H]2CN(C(=O)OC)CCO2)c(F)c1]

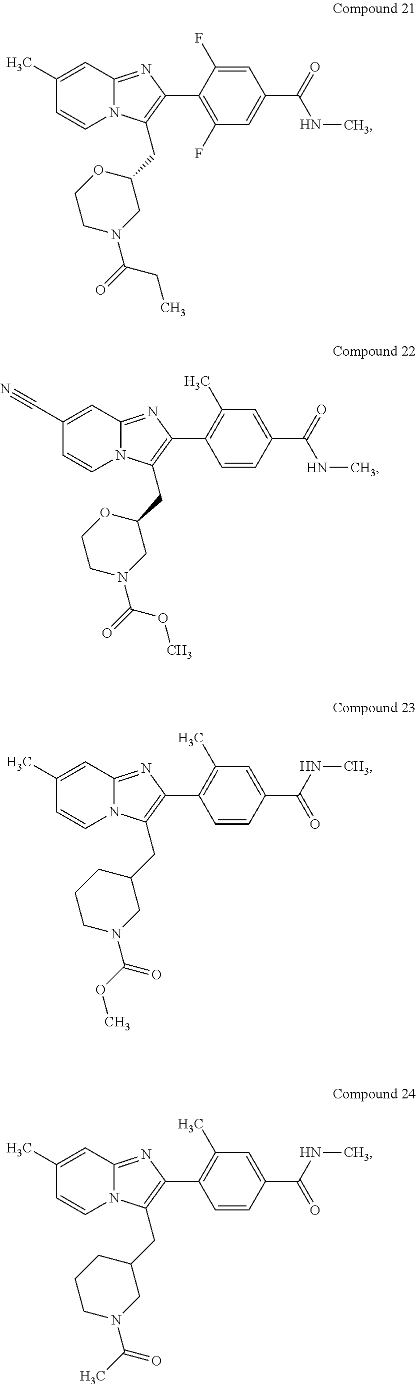 [SMILES: CCC(=O)N1CCO[C@H](Cc2c(-c3c(F)cc(C(=O)NC)cc3F)nc3cc(C)ccn23)C1 ; CNC(=O)c1ccc(-c2nc3cc(C#N)ccn3c2C[C@H]2CN(C(=O)OC)CCO2)c(C)c1 ; CNC(=O)c1ccc(-c2nc3cc(C)ccn3c2CC2CCCN(C(=O)OC)C2)c(C)c1 ; CNC(=O)c1ccc(-c2nc3cc(C)ccn3c2CC2CCCN(C(C)=O)C2)c(C)c1]

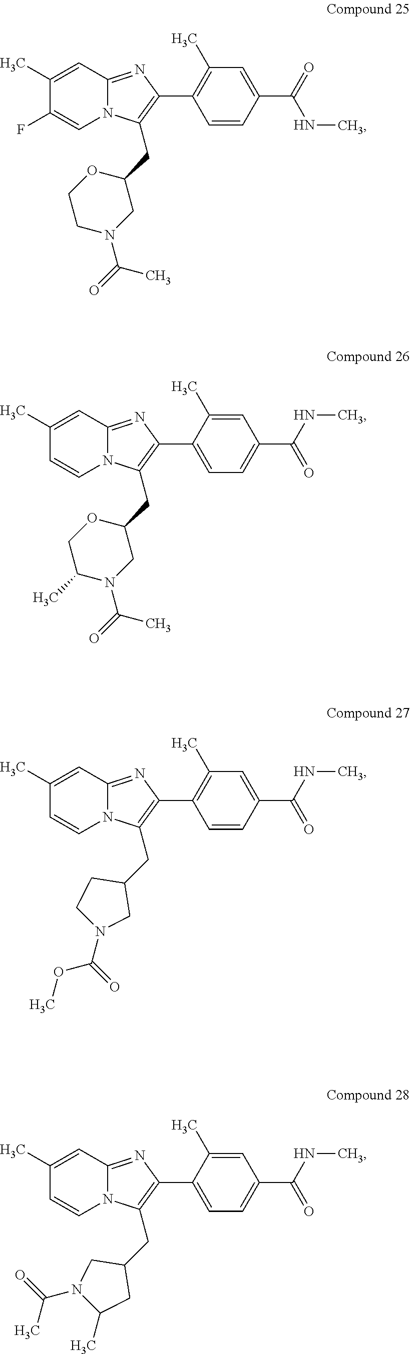 [SMILES: CNC(=O)c1ccc(-c2nc3cc(C)c(F)cn3c2C[C@H]2CN(C(C)=O)CCO2)c(C)c1 ; CNC(=O)c1ccc(-c2nc3cc(C)ccn3c2C[C@H]2CN(C(C)=O)[C@H](C)CO2)c(C)c1 ; CNC(=O)c1ccc(-c2nc3cc(C)ccn3c2CC2CCN(C(=O)OC)C2)c(C)c1 ; CNC(=O)c1ccc(-c2nc3cc(C)ccn3c2CC2CC(C)N(C(C)=O)C2)c(C)c1]

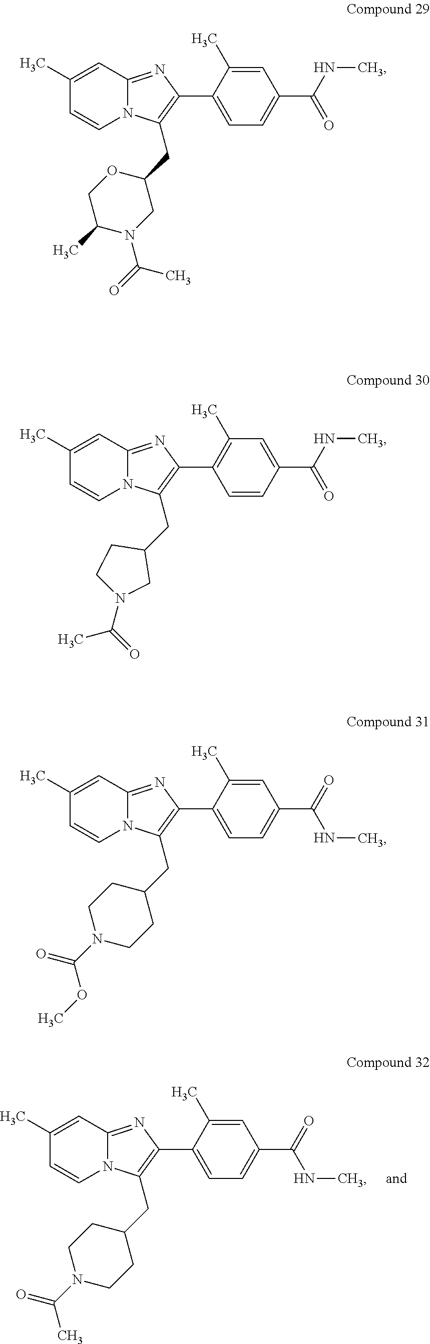 [SMILES: CNC(=O)c1ccc(-c2nc3cc(C)ccn3c2C[C@H]2CN(C(C)=O)[C@@H](C)CO2)c(C)c1 ; CNC(=O)c1ccc(-c2nc3cc(C)ccn3c2CC2CCN(C(C)=O)C2)c(C)c1 ; CNC(=O)c1ccc(-c2nc3cc(C)ccn3c2CC2CCN(C(=O)OC)CC2)c(C)c1 ; CNC(=O)c1ccc(-c2nc3cc(C)ccn3c2CC2CCN(C(C)=O)CC2)c(C)c1]

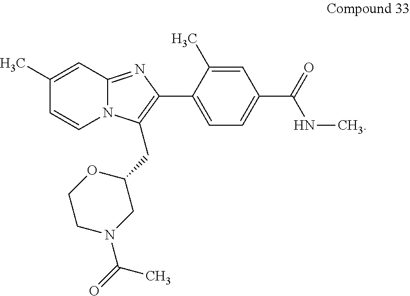 [SMILES: CNC(=O)c1ccc(-c2nc3cc(C)ccn3c2C[C@@H]2CN(C(C)=O)CCO2)c(C)c1]

In some embodiments of the methods described herein, the P2X3 antagonist is a compound of Formula (I), or a pharmaceutically acceptable salt thereof, wherein the compound of Formula (I) corresponds in structure to

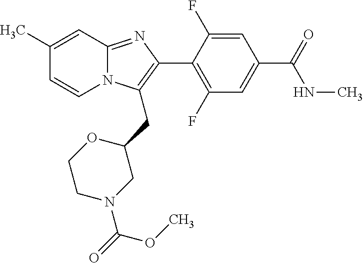

In some embodiments of the methods described herein, the P2X3 antagonist is a compound of Formula (I), or a pharmaceutically acceptable salt thereof, wherein the compound of Formula (I) corresponds in structure to

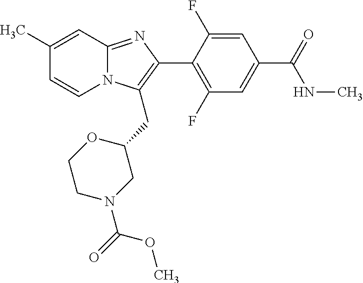

In some embodiments of the methods described herein, the P2X3 antagonist is a compound of Formula (I), or a pharmaceutically acceptable salt thereof, wherein the compound of Formula (I) corresponds in structure to

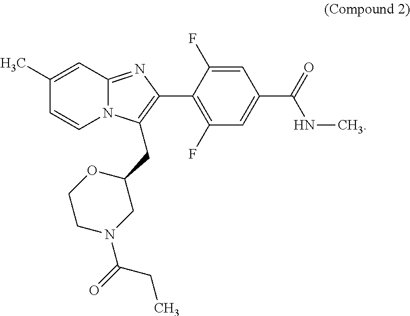 [SMILES: CCC(=O)N1CCO[C@@H](Cc2c(-c3c(F)cc(C(=O)NC)cc3F)nc3cc(C)ccn23)C1]

In some embodiments of the methods described herein, the P2X3 antagonist is a compound of Formula (I), or a pharmaceutically acceptable salt thereof, wherein the compound of Formula (I) corresponds in structure to

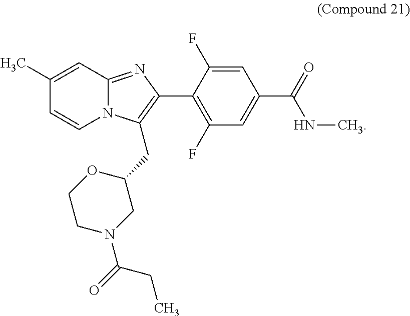 [SMILES: CCC(=O)N1CCO[C@H](Cc2c(-c3c(F)cc(C(=O)NC)cc3F)nc3cc(C)ccn23)C1]

In some embodiments of the methods described herein, the P2X3 antagonist corresponds in structure to

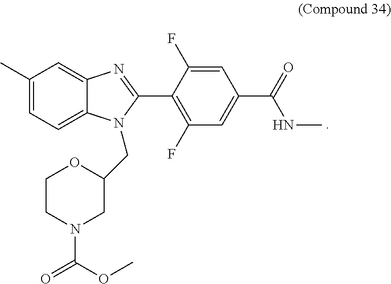

In some embodiments of the methods described herein, the P2X3 antagonist corresponds in structure to

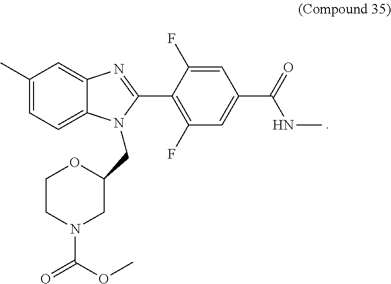

In some embodiments of the methods described herein, the P2X3 antagonist corresponds in structure to

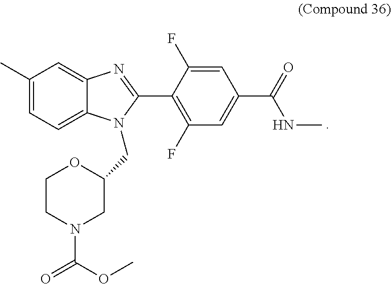

In some embodiments of the methods described herein, is a method of treating renal pruritus. In some embodiments of the methods described herein, is a method of treating cholestatic pruritus. In some embodiments of the methods described herein, is a method of treating hematologic pruritus. In some embodiments of the methods described herein, is a method of treating endocrine pruritus. In some embodiments of the methods described herein, is a method of treating pruritus related to malignancy. In some embodiments of the methods described herein, is a method of treating idiopathic generalized pruritus.

In some embodiments of the methods described herein, the pruritus is associated with a primary skin disorder. In some embodiments of the methods described herein, the pruritus is associated with a primary skin disorder selected from the group consisting of xerosis, atopic dermatitis, urticaria, psoriasis, arthropod assault, mastocytosis, dermatitis herpetiformis, and pemphigoid. In some embodiments of the methods described herein, the pruritus is associated with xerosis. In some embodiments of the methods described herein, the pruritus is associated with atopic dermatitis. In some embodiments of the methods described herein, the pruritus is associated with urticaria. In some embodiments of the methods described herein, the pruritus is associated with psoriasis. In some embodiments of the methods described herein, the pruritus is associated with arthropod assault. In some embodiments of the methods described herein, the pruritus is associated with mastocytosis. In some embodiments of the methods described herein, the pruritus is associated with dermatitis herpetiformis. In some embodiments of the methods described herein, the pruritus is associated with pemphigoid.

In some embodiments of the methods described herein, the pruritus is an acute condition. In some embodiments of the methods described herein, the pruritus is a chronic condition.

In certain embodiments, a disclosed compound utilized by one or more of the foregoing methods is one of the generic, subgeneric, or specific compounds described herein, such as a compound of Formula (I) described herein.

### Preparation of the Compounds

The compounds used in the methods described herein are made according to procedures disclosed in U.S. Pat. No. 9,598,409, which is herein incorporated by reference in its entirety, or by known organic synthesis techniques, starting from commercially available chemicals and/or from compounds described in the chemical literature. Commercially available chemicals are obtained from standard commercial sources including Acros Organics (Geel, Belgium), Aldrich Chemical (Milwaukee, Wis., including Sigma Chemical and Fluka), Apin Chemicals Ltd. (Milton Park, UK), Ark Pharm, Inc. (Libertyville, Ill.), Avocado Research (Lancashire, U.K.), BDH Inc. (Toronto, Canada), Bionet (Cornwall, U.K.), Chemservice Inc. (West Chester, Pa.), Combi-blocks (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Spectrum Chemicals (Gardena, Calif.), Sundia Meditech, (Shanghai, China), TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁶O, ¹⁷O, ³¹P, ³²P, ³⁵S, ¹⁸F, and ³⁶Cl, respectively. Compounds described herein, and the pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Prodrugs

In some embodiments, the compounds described herein are formulated as agents which are converted in vivo to active forms in order to alter the biodistribution or the pharmacokinetics for a particular agent. For example, a carboxylic acid group can be esterified, e.g., with a methyl group or an ethyl group to yield an ester. When the ester is administered to a subject, the ester is cleaved, enzymatically or non enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids. Alternatively, other functional groups may be modified into a prodrug form. For instance, an amine group may be converted into a carbamate or amide which would be cleavable in vivo.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Pharmaceutical Compositions

In certain embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington; The Science and Practice of Pharmacy (Gennaro, 21^{st} Ed. Mack Pub. Co., Easton, Pa. (2005)).

Accordingly, provided herein is a pharmaceutical composition comprising at least one compound described herein, or a pharmaceutically acceptable salt, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable carrier and a compound of Formula (I), or a pharmaceutically acceptable salt thereof.

Another embodiment provides a pharmaceutical composition consisting essentially of a pharmaceutically acceptable carrier and a compound of Formula (I), or a pharmaceutically acceptable salt thereof.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, topical, buccal, parenteral (e.g., subcutaneous, intramuscular, intradermal, or intravenous), or aerosol administration.

Exemplary pharmaceutical compositions are used in the form of a pharmaceutical preparation, for example, in solid, semisolid or liquid form, which includes one or more of a disclosed compound, as an active ingredient, in a mixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

In some embodiments, a compound of Formula (I) described herein are administered to subjects in a biologically compatible form suitable for topical administration to treat or prevent dermal diseases, disorders or conditions. By “biologically compatible form suitable for topical administration” is meant a form of the compound of Formula (I) to be administered in which any toxic effects are outweighed by the therapeutic effects of the inhibitor. Administration of a compound of Formula (I) as described herein can be in any pharmacological form including a therapeutically effective amount of a compound of Formula (I) alone or in combination with a pharmaceutically acceptable carrier.

Topical administration of a compound of Formula (I) may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Chapter 67 of Remington's Pharmaceutical Sciences, 15th Edition (1975) published by Mack Publishing Company.

Dermal or skin patches are another method for transdermal delivery of the therapeutic or pharmaceutical compositions described herein. Patches can provide an absorption enhancer such as DMSO to increase the absorption of the compounds. Patches can include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin.

The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

In one embodiment, the topical composition may, for example, take the form of hydrogel based on polyacrylic acid or polyacrylamide; as an ointment, for example with polyethyleneglycol (PEG) as the carrier, like the standard ointment DAB 8 (50% PEG 300, 50% PEG 1500); or as an emulsion, especially a microemulsion based on water-in-oil or oil-in-water, optionally with added liposomes. Suitable permeation accelerators (entraining agents) include sulphoxide derivatives such as dimethylsulfoxide (DMSO) or decylmethylsulfoxide (decyl-MSO) and transcutol (diethyleneglycolmonoethylether) or cyclodextrin; as well as pyrrolidones, for example 2-pyrrolidone, N-methyl-2-pyrrolidone, 2-pyrrolidone-5-carboxylic acid, or the biodegradable N-(2-hydroxyethyl)-2-pyrrolidone and the fatty acid esters thereof; urea derivatives such as dodecylurea, 1,3-didodecylurea, and 1,3-diphenylurea; terpenes, for example D-limonene, menthone, a-terpinol, carvol, limonene oxide, or 1,8-cineol.

Ointments, pastes, creams and gels also can contain excipients, such as starch, tragacanth, cellulose derivatives, polyethylene glycols, silicones, bentonites, silicic acid, and talc, or mixtures thereof. Powders and sprays also can contain excipients such as lactose, talc, silicic acid, aluminum hydroxide, calcium silicates and polyamide powder, or mixtures of these substances. Solutions of nanocrystalline antimicrobial metals can be converted into aerosols or sprays by any of the known means routinely used for making aerosol pharmaceuticals. In general, such methods comprise pressurizing or providing a means for pressurizing a container of the solution, usually with an inert carrier gas, and passing the pressurized gas through a small orifice. Sprays can additionally contain customary propellants, such a chlorofluorohydrocarbons and volatile unsubstituted hydrocarbons, such as butane and propane.

In some embodiments for preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, cellulose, microcrystalline cellulose, silicified microcrystalline cellulose, lactose, sucrose, glucose, mannitol, and/or silicic acid; (2) binders, such as, for example, carboxymethylcellulose, hypromellose, alginates, gelatin, polyvinyl pyrrolidone, sucrose and/or acacia; (3) humectants, such as glycerol; (4) disintegrating agents, such as crospovidone, croscarmellose sodium, sodium starch glycolate, agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, docusate sodium, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, in some embodiments, the compositions comprise buffering agents. In some embodiments, solid compositions of a similar type are also employed as fillers in soft and hard-filled gelatin capsules using such excipients as lactose or milk sugars, as well as high molecular weight polyethylene glycols and the like.

In some embodiments, a tablet is made by compression or molding, optionally with one or more accessory ingredients. In some embodiments, compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. In some embodiments, molded tablets are made by molding in a suitable machine a mixture of the subject composition moistened with an inert liquid diluent. In some embodiments, tablets, and other solid dosage forms, such as dragees, capsules, pills and granules, are scored or prepared with coatings and shells, such as enteric coatings and other coatings.

Compositions for inhalation or insufflation include solutions and suspensions in pharmaceutically acceptable, aqueous or organic solvents, or mixtures thereof, and powders.

Liquid dosage forms for oral administration include pharmaceutically acceptable emulsions, microemulsions, solutions, suspensions, syrups and elixirs. In addition to the subject composition, in some embodiments, the liquid dosage forms contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

In some embodiments, suspensions, in addition to the subject composition, contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, powders and sprays contain, in addition to a subject composition, excipients such as lactose, talc, silicic acid, aluminum hydroxide, calcium silicates and polyamide powder, or mixtures of these substances. In some embodiments, sprays additionally contain customary propellants, such as chlorofluorohydrocarbons and volatile unsubstituted hydrocarbons, such as butane and propane.

Compositions and compounds disclosed herein alternatively are administered by aerosol. This is accomplished by preparing an aqueous aerosol, liposomal preparation or solid particles containing the compound. In some embodiments, a non-aqueous (e.g., fluorocarbon propellant) suspension is used. In some embodiments, sonic nebulizers are used because they minimize exposing the agent to shear, which results in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which, in some embodiments, contain antioxidants, buffers, bacteriostats, solutes which render the formulation isotonic with the blood of the intended recipient or suspending or thickening agents.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants

The dose of the composition comprising at least one compound described herein differs, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. In some embodiments, the optimal dose depends upon the body mass, weight, or blood volume of the patient.

Oral doses typically range from about 1.0 mg to about 1000 mg, one to four times, or more, per day.

### Pruritus Conditions:

In some embodiments of the methods of treating pruritus described herein, the pruritus is associated with an inflammatory skin disease. In some embodiments, the inflammatory skin disease includes, but is not limited to, atopic dermatitis, allergic, irritant contact dermatitis, exsiccation dermatitis, nummular and dyshidrotic dermatitis, lichen planus, lichen sclerosus et atrophicus, polymorphous light eruption psoriasis, Grover's disease, mucinosis, mastocytosis, and urticaria;

In some embodiments of the methods of treating pruritus described herein, the pruritus is associated with an infectious skin disease. In some embodiments, the infectious skin disease includes, but is not limited to, mycoses, bacterial and viral infections, scabies, pediculosis, insect bites, and folliculitides.

In some embodiments of the methods of treating pruritus described herein, the pruritus is associated with an autoimmune skin disease. In some embodiments, the autoimmune skin disease includes, but is not limited to, Bullous skin disorders, dermatitis herpetiformis (Duhring's disease), bullous pemphigoid, genodermatoses such as Darier's disease, and Hailey-Hailey disease.

In some embodiments of the methods of treating pruritus described herein, the pruritus is associated with a pregnancy-related skin disease. In some embodiments, the pregnancy-related skin disease includes, but is not limited to, polymorphic eruption of pregnancy (PEP, formerly known as PUPPP), atopic eruption of pregnancy, pemphigoid gestationis, and neoplasias such as cutaneous T-cell lymphoma.

Prurigo nodularis (PN), or nodular prurigo, is a particularly severe form of chronic itching that may treated by methods and compositions of the present invention. Characterized by itchy, excoriated, lichenified papules and nodules, PN can occur at any age, but most often presents in middle-aged and elderly patients on their arms and legs (E. Weisshaar and S. Stander, Acta Derm. Venereol., 2012, 92:532-533). PN may result in permanent changes to the skin, including nodular lichenification, hyperkeratosis, hyperpigmentation, and skin thickening.

Uremic pruritus is a common and disturbing problem affecting chronic kidney disease patients undergoing dialysis that may be treated by methods and compositions of the present invention. Uremic pruritus has a major clinical impact because it is strongly associated with poor quality of life, impaired sleep and depression.

In some embodiments, examples of pruritus-associated conditions include without limitation: dermatological disorders and conditions (including inflammatory and non-inflammatory skin conditions), including but not limited to adult blaschkitis, amyloidoses (e.g., primary cutaneous amyloidosis [including macular amyloidosis, lichen amyloidosis and nodular amyloidosis]), burns (e.g., chemical burns and sunburn), dermatitis (e.g., atopic dermatitis, contact dermatitis (including allergic contact dermatitis, irritant contact dermatitis and photodermatitis), eczema (e.g., autosensitization dermatitis, dermatitis herpetiformis [Duhring's disease], discoid eczema, dyshidrosis [pompholyx], hand eczema, id reaction [generalized eczema], nummular eczema, stasis dermatitis [gravitational eczema], venous eczema and xerotic eczema), pustular dermatitis (e.g., eosinophilic pustular folliculitis [Ofuji's disease], reactive arthritis [Reiter's disease] and subcorneal pustular dermatosis [Sneddon-Wilkinson disease]), and seborrheic dermatitis (e.g., infantile seborrheic dermatitis, Leiner's disease and pityriasis simplex capillitii [dandruff])}, erythroderma (exfoliative dermatitis), folliculitis, pseudofolliculitis barbae (barber's itch), hidradenitis suppurativa, ichthyoses (e.g., ichthyosis vulgaris, congenital ichthyosis, epidermolytic hyperkeratosis and lamellar ichthyosis), lichen planus (e.g., cutaneous lichen planus and oral lichen planus), lichen sclerosis (e.g., lichen sclerosis et atrophicus of the vulva), lichen simplex (e.g., lichen simplex chronicus [neurodermatitis]), linear IgA bullous dermatosis (linear IgA dermatosis), lupus erythematosus (e.g., cutaneous lupus erythematosus, discoid lupus erythematosus and systemic lupus erythematosus), miliaria (sweat rash), palmoplantar keratoderma (e.g., punctate palmoplantar keratoderma), pityriasis (e.g., pityriasis amiantacea, pityriasis lichenoides [including pityriasis lichenoides chronica and pityriasis lichenoides et varioliformis acuta], pityriasis rosea, pityriasis rubra pilaris [Devergie's disease] and pityriasis versicolor), prurigo (e.g., actinic prurigo, Besnier's prurigo, prurigo nodularis, prurigo pigmentosa and prurigo simplex), pruritus ani, pruritus scroti, pruritus vulvae, psoriasis (e.g., erythrodermic psoriasis, Guttate psoriasis [eruptive psoriasis], psoriasis vulgaris [chronic stationary psoriasis], pustular psoriasis, and pustulosis palmaris et plantaris), parapsoriasis (e.g., large plaque parapsoriasis and small plaque parapsoriasis [chronic superficial dermatitis]), puncta pruritica (itchy points), rashes (e.g., intertrigo and perioral dermatitis), rosacea, urticaria (e.g., contact urticaria [including hives] and idiopathic urticaria), vitiligo, xerosis (dry skin), chapped skin (e.g., chapped feet), scalp pruritus, scab healing, scar development, and development of moles, pimples and ingrown hair; medical disorders and conditions (including peripheral and systemic disorders), including but not limited to atopic diathesis, autoimmune disorders (e.g., celiac disease, dermatomyositis, Graves' disease, pemphigoid [e.g., bullous pemphigoid], scleroderma and Sjogren's syndrome), blood disorders (e.g., anemia [e.g., iron deficiency anemia and sickle cell anemia], hypercalcemia, myelodysplastic syndromes and polycythemia [e.g., polycythemia vera]), Creutzfeldt-Jakob disease (e.g., prion pruritus), diabetes mellitus, genetic diseases (e.g., Alagille syndrome, Darier's disease, epidermolysis bullosa, Hailey-Hailey disease and Sjogren-Larsson syndrome), Grover's disease, HIV/AIDS, kidney disorders (e.g., diabetic nephropathy, glomerulonephritis, chronic kidney disease, end-stage kidney disease and chronic kidney failure), uraemia (e.g., uremic pruritus [renal pruritus]), liver diseases (e.g., cirrhosis [e.g., primary biliary cirrhosis], hepatitis [including hepatitis A, B, C, D and E and their chronic conditions], and liver failure), cholestasis (e.g., cholestatic pruritus), jaundice (e.g., biliary pruritus), lymphadenopathy (e.g., enlarged lymph nodes), mast cell diseases (e.g., mast cell activation syndrome and mastocytosis), multiple sclerosis, neuropathies (e.g., peripheral neuropathy [e.g., brachioradial pruritus, notalgia paresthetica, polyneuropathy and small fiber peripheral neuropathy]), nerve irritation, pinched nerves, parathyroid disorders (e.g., hyperparathyroidism and hypoparathyroidism), thyroid disorders (e.g., hyperthyroidism, hypothyroidism and myxedema), stroke, cancers (e.g., carcinoid syndrome, leukemia (e.g., leukemia cutis and lymphatic leukemia), lymphomas (e.g., Hodgkin's disease and non-Hodgkin lymphomas [e.g., cutaneous B-cell lymphoma and cutaneous T-cell lymphoma (including mycosis fungoides and Sezary's disease)]), Kaposi's sarcoma, multiple myeloma and skin cancers}, tumors (e.g., brain tumor, plasmacytoma, and solid tumors of the cervix, colon and prostate), paraneoplastic pruritus, psychiatric disorders (e.g., stress, anxiety disorders, delusional parasitosis, depression, obsessive-compulsive disorders [e.g., neurotic excoriation], and tactile hallucinations), aging (e.g., senile pruritus) and changes in hormonal balances associated with aging (e.g., perimenopause and menopause); infections and infestations, including but not limited to cercarial dermatitis (swimmer's itch), insect bites and stings (e.g., by ants, bees, chiggers, fleas, lice [including body lice, head lice and pubic lice], mites, mosquitos, spiders, ticks and wasps), scabies, bacterial infections (e.g., abscess, dermatitis gangrenosa, ecthyma, erythrasma, impetigo and Lyme disease), fungal infections (e.g., candidiasis, dermatophytosis, tinea corporis [ringworm of the body], tinea cruris [jock itch] and tinea pedis [athlete's foot]), viral infections (e.g., herpes (including herpes zoster [shingles] and post-herpetic itch), measles, parvovirus infections (e.g., parvovirus B19), varicella (chickenpox) and Yellow fever}, and worm infections (e.g., helminths (e.g., helminthiasis [helminthosis]), hookworms (e.g., cutaneous larva migrans), Onchocerca worms (e.g., onchocerciasis [river blindness]), pinworms, roundworms (e.g., filariasis and trichinosis) and Schistosoma worms (e.g., schistosomiasis)}; reactions to allergens and irritants, including but not limited to allergic rhinitis (e.g., pollinosis [including hay fever]), asthma, animal allergens (e.g., cat dander and dog dander), chemical allergens (e.g., acids [e.g., abietic acid and sorbic acid], cosmetics, detergents, dyes, fabric softeners, fungicides, hydroxy ethyl starch and latex), food allergens (e.g., milk proteins, peanuts, tree nuts, seafood, spices, preservatives [e.g., nitrates], vitamins [e.g., vitamins A and B], alcohol, caffeine and monosodium glutamate), metal and metal salt allergens (e.g., chromium, cobalt, gold and nickel and salts thereof), plant allergens (e.g., Balsam of Peru and urushiol [e.g., in poison ivy, poison oak and poison sumac]), chemical irritants (e.g., acids, alkalis, metalworking fluids, solvents, surfactants, detergents, soaps, cleaning products, cosmetics, perfumes, deodorants, antiperspirants, food flavorings, spices, preservatives [e.g., formaldehyde and parabens], monomers and polymers [e.g., acrylics, epoxy resins, ethylene oxide, latex and lacquers], and oils [e.g., kerosene]), fabrics (e.g., wool), plant irritants (e.g., alkyl resorcinols [e.g., in Grevillea banksii, Grevillea “Robyn Gordon” and Gingko biloba]), and physical irritants (e.g., water [e.g., aquadynia and aquagenic pruritus), low humidity from air conditioning, and cold temperature); pruritus caused by drugs/medication, including but not limited to chloroquine, hydroxyethyl cellulose, hydroxyethyl starch, angiotensin-converting enzyme inhibitors, xanthine oxidase inhibitors (e.g., allopurinol), antibiotics (e.g., isoniazid, neomycin, penicillin, sulfonamides and vancomycin), antifungals (e.g., fluconazole, griseofulvin, itraconazole and ketoconazole), neuroleptics/antipsychotics (e.g., phenothiazines), antiarrhythmic drugs (e.g., amiodarone and quinidine), chemotherapeutic drugs, diuretic drugs (e.g., hydrochlorothiazide), statins (e.g., simvastatin), and drugs (e.g., opioids) that activate the histamine H.sub.l receptor or trigger histamine release; and conditions related to pregnancy, including but not limited to gestational pemphigoid, impetigo herpetiformis, intrahepatic cholestasis of pregnancy (pruritus gravidarum), polymorphic eruption of pregnancy, prurigo of pregnancy, pruritic folliculitis of pregnancy, and pruritic urticarial papules and plaques of pregnancy.

In some embodiments is a method of treating pruritus in a mammal wherein the pruritus is associated with a kidney disease or a therapeutic procedure to treat a kidney disease. In some embodiments is a method of treating pruritus in a mammal wherein the pruritus is associated with a kidney disease. In some embodiments is a method of treating pruritus in a mammal wherein the pruritus is associated with a chronic kidney disease. In some embodiments is a method of treating pruritus in a mammal wherein the pruritus is associated with a therapeutic procedure to treat a kidney disease. In some embodiments is a method of treating pruritus in a mammal wherein the pruritus is associated with a therapeutic procedure to treat a kidney disease, wherein the therapeutic procedure to treat the kidney disease is hemodialysis or peritoneal dialysis. In some embodiments is a method of treating pruritus in a mammal wherein the pruritus is associated with a therapeutic procedure to treat a kidney disease, wherein the therapeutic procedure to treat the kidney disease is hemodialysis. In some embodiments is a method of treating pruritus in a mammal wherein the pruritus is associated with a therapeutic procedure to treat a kidney disease, wherein the therapeutic procedure to treat the kidney disease is peritoneal dialysis. In some embodiments is a method of treating pruritus in a mammal wherein the pruritus is associated with a medical procedure or treatment. In some embodiments is a method of treating pruritus in a mammal wherein the pruritus is associated with a medical procedure. In some embodiments is a method of treating pruritus in a mammal wherein the pruritus is associated with a medical treatment. In some embodiments is a method of treating pruritus in a mammal wherein the pruritus is associated with a medical treatment with a drug selected from the group consisting of opioids, anti-malarial drugs, anti-cancer therapies, and epidermal growth factor receptor inhibitors. In some embodiments is a method of treating pruritus in a mammal wherein the pruritus is associated with a medical treatment with opioids.

In some embodiments is a method of treating pruritus in a mammal wherein the pruritus is associated with a medical treatment with anti-malarial drugs. In some embodiments is a method of treating pruritus in a mammal wherein the pruritus is associated with a medical treatment with anti-cancer therapies. In some embodiments is a method of treating pruritus in a mammal wherein the pruritus is associated with a medical treatment with epidermal growth factor receptor inhibitors.

### Pharmaceutical Combinations

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is effected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

In some embodiments is a method of treating pruritus in a mammal in need thereof, the method comprising administering to the mammal a P2X3 antagonist further comprising administering to the mammal one or more additional pharmaceutical agents. In some embodiments is a method of treating pruritus in a mammal in need thereof, the method comprising administering to the mammal a compound of Formula (I) further comprising administering to the mammal one or more additional pharmaceutical agents. In some embodiments, the one or more additional pharmaceutical agents are selected from the group consisting of antihistamines, including but not limited to antihistamines that inhibit action at the histamine Hi receptor (e.g., acrivastine, antazoline, azelastine, bilastine, brompheniramine, buclizine, bromodiphenhydramine, carbinoxamine, cetirizine, chlorpromazine, cyclizine, chlorpheniramine, chlorodiphenhydramine, clemastine, cyproheptadine, desloratadine, dexbrompheniramine, dexchlorpheniramine, dimenhydrinate, dimetindene, diphenhydramine, doxepin, doxylamine, ebastine, embramine, fexofenadine, hydroxyzine, levocetirizine, loratadine, meclozine, mepyramine, mirtazapine, olopatadine, orphenadrine, phenindamine, pheniramine, phenyltoloxamine, promethazine, pyrilamine, quetiapine, rupatadine, tripelennamine and triprolidine), and antihistamines that inhibit action at the histamine H₄ receptor (e.g., thioperamide, JNJ 7777120 and VUF-6002), and analogs and derivatives thereof; serotonin receptor antagonists, including but not limited to 5-HT₂ antagonists (e.g., clozapine, cyproheptadine, ketanserin, pizotifen and quetiapine) and 5-HT₃ antagonists (e.g., alosetron, cilansetron, dolasetron, granisetron, ondansetron, palonosetron and tropisetron), and analogs and derivatives thereof; neurokinin-1 (NK-1) receptor antagonists, including but not limited to serlopitant, aprepitant, casopitant (GW679769), dapitant, ezlopitant, fosaprepitant, lanepitant (LY-303870), maropitant, netupitant, nolpitant, orvepitant, rolapitant, vestipitant, vofopitant, AV-818, BIIF 1149CL, CP122,721, DNK-333, GSK-424887, L-733060, L-759274, LY-686017, M516102, and TA-5538, and analogs and derivatives thereof; opioid receptor antagonists, including but not limited to butorphanol, cyprodime, levallorphan (lorfan or naloxiphan), nalbuphine, nalorphine (lethidrone or nalline), naloxone, naloxol, nalmefene, naltrexone (e.g., naltrexone 1% cream) and naltrexol, and analogs and derivatives thereof; opioid receptor agonists, including but not limited to selective kappa opioid receptor agonists (e.g., asimadoline, bremazocine, dynorphin, enadoline, ketazocine, nalfurafine, salvinorin A, 2-methoxymethyl salvinorin B, 2-ethoxymethyl salvinorin B, 2-fluoroethoxymethyl salvinorin B, spiradoline, tifluadom, BRL-52537, FE 200665, GR-89696, HZ-2, ICI-199,441, ICI-204,448, LPK-26, U-50488 and U-69,593), and analogs and derivatives thereof; Janus kinase (JAK) inhibitors, including but not limited to JAK1 inhibitors (e.g., GLPG0634 and GSK2586184), JAK2 inhibitors (e.g., lestaurtinib, pacritinib, CYT387 and TG101348), JAK1/JAK2 inhibitors (e.g., baricitinib and ruxolitinib), and JAK3 inhibitors (e.g., tofacitinib), and analogs and derivatives thereof; immunomodulators and immunosuppressants, including but not limited to thalidomide, antimetabolites (e.g., antifolates such as methotrexate), and calcineurin inhibitors (e.g., ciclosporin [cyclosporin], pimecrolimus and tacrolimus), and analogs and derivatives thereof; antidepressants, including but not limited to tricyclic antidepressants (e.g., amitriptyline, amitriptylinoxide, amoxapine, dosulepin [dothiepin], doxepin and melitracen), tetracyclic antidepressants (e.g., amoxapine, maprotiline, mazindol, mianserin, mirtazapine and setiptiline), selective serotonin reuptake inhibitors (SSRIs, e.g., citalopram, dapoxetine, escitalopram, fluoxetine, fluvoxamine, paroxetine and sertraline), and serotonin-norepinephrine reuptake inhibitors (SNRIs, e.g., bicifadine, duloxetine, milnacipran, levomilnacipran, sibutramine, venlafaxine, desvenlafaxine and SEP-227162), and analogs and derivatives thereof; anticonvulsants, including but not limited to carbamazepine, gabapentin, pregabalin, and valproic acid and salts thereof (e.g., sodium valproate), and analogs and derivatives thereof; corticosteroids, including but not limited to hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], and mometasone and derivatives thereof [e.g., mometasone furoate]), acetonides and related substances (e.g., amcinonide, budesonide, ciclesonide, desonide, fluocinonide, fluocinolone acetonide, flurandrenolide [flurandrenolone or fludroxycortide], halcinonide, triamcinolone acetonide and triamcinolone alcohol), and carbonates (e.g., prednicarbate), and analogs and derivatives thereof; local anesthetics, including but not limited to amides (e.g., articaine, bupivacaine, cinchocaine [dibucaine], etidocaine, levobupivacaine, lidocaine [e.g., lidocaine 2.5-5% cream], prilocaine [e.g., prilocaine 2.5% cream], EMLA [lidocaine 2.5%/prilocaine 2.5% cream], mepivacaine, ropivacaine and trimecaine), esters (e.g., benzocaine, chloroprocaine, cocaine, cyclomethycaine, dimethocaine [larocaine], piperocaine, procaine [novocaine], proparacaine, propoxycaine, stovaine and tetracaine [amethocaine]), ethers (e.g., polidocanol [e.g., polidocanol 3% foam] and pramocaine [pramoxine] [e.g., pramoxine 1% cream]), and naturally derived local anesthetics (e.g., cocaine, eugenol, menthol, saxitoxin, neosaxitoxin and tetrodotoxin), and analogs and derivatives thereof; counterirritants and cooling agents, including but not limited to capsaicin, camphor, mint oil, menthol (e.g., menthol 1-3% cream), and phenol (e.g., in calamine lotion), and analogs and derivatives thereof; moisturizers, including but not limited to aqueous moisturizers, low pH moisturizers containing an acid (e.g., lactic acid), and moisturizers containing a humectant that attracts and retains water (e.g., glycerol, sorbitol, lactate, urea, and hyaluronic acid and salts thereof), an occlusive that prevents evaporation (e.g., oils (e.g., mineral oil and silicone oil [e.g., dimethicone]) and petroleum jelly (petrolatum)}, and/or an emollient that provides partial hydration and occlusion (e.g., oils, waxes [e.g., lanolin and paraffin], lipids [e.g., phospholipids, ceramides, triglycerides, glycol stearate, glyceryl stearate, fatty acids and squalene], and sterols [e.g., cholesterol and phytosterol]), and analogs and derivatives thereof; and other kinds of antipruritic agents, including but not limited to S-adenosyl methionine, botulinum toxin (e.g., botulinum toxin types A and B), vitamin D and analogs and derivatives thereof (e.g., calcitriol and calcipotriol [calcipotriene]), non-steroidal anti-inflammatory drugs (NSAIDs, e.g., aspirin), cannabinoid receptor agonists (e.g., CB₂ agonists, such as palmitoylethanolamide), inhibitors of cytokines (e.g., antibodies to interleukins, such as IL-31), antagonists of the prostaglandin D₂ receptor (DPi) and/or the chemoattractant receptor homologous molecule expressed on TH₂ cells (CRTH2) (e.g., TS-022), phosphodiesterase (PDE) inhibitors (e.g., PDE4 inhibitors, such as apremilast), protease-activated receptor 2 (PAR2) antagonists (e.g., GB83), transient receptor potential vanilloid (TRPV) antagonists (e.g., TRPV1 antagonists, such as capsazepine and SB-705498), inhibitors of neurotrophic tyrosine kinase receptors (e.g., TrkA inhibitors, such as CT327), antimicrobials (including antibiotics, antifungals, antivirals and antiparasitics, such as crotamiton and rifampin [rifampicin]), bile absorption-reducing or bile sequestering agents (e.g., ursodeoxycholic acid [ursodiol]), ultraviolet radiation (e.g., ultraviolet A and B), and therapeutic agents that treat the underlying causes of the pruritus-associated conditions, and analogs and derivatives thereof.

In some embodiments is a method of treating pruritus in a mammal in need thereof, the method comprising administering to the mammal a P2X3 antagonist further comprising administering to the mammal an NK-1 antagonist. In some embodiments is a method of treating pruritus in a mammal in need thereof, the method comprising administering to the mammal a compound of Formula (I), or a pharmaceutically acceptable salt thereof, further comprising administering to the mammal an NK-1 antagonist wherein the NK-1 antagonist is selected from the group consisting of, but not limited to serlopitant, aprepitant, casopitant, dapitant, ezlopitant, fosaprepitant, lanepitant, maropitant, netupitant, nolpitant, orvepitant, rolapitant, vestipitant, vofopitant, AV-818, BIIF 1149CL, CP122,721, DNK-333, GSK-424887, L-733060, L-759274, LY-686017, M516102, and TA-5538, and analogs and derivatives thereof. In some embodiments is a method of treating pruritus in a mammal in need thereof, the method comprising administering to the mammal a compound of Formula (I), or a pharmaceutically acceptable salt thereof, further comprising administering to the mammal an NK-1 antagonist wherein the NK-1 antagonist is selected from the group consisting of serlopitant, orvepitant, rolapitant, aprepitant, and fosaprepitant, or a pharmaceutically acceptable salt thereof. In some embodiments is a method of treating pruritus in a mammal in need thereof, the method comprising administering to the mammal a compound of Formula (I), or a pharmaceutically acceptable salt thereof, further comprising administering to the mammal an NK-1 antagonist wherein the NK-1 antagonist is serlopitant, or a pharmaceutically acceptable salt thereof. In some embodiments is a method of treating pruritus in a mammal in need thereof, the method comprising administering to the mammal a compound of Formula (I), or a pharmaceutically acceptable salt thereof, further comprising administering to the mammal an NK-1 antagonist wherein the NK-1 antagonist is orvepitant, or a pharmaceutically acceptable salt thereof. In some embodiments is a method of treating pruritus in a mammal in need thereof, the method comprising administering to the mammal a compound of Formula (I), or a pharmaceutically acceptable salt thereof, further comprising administering to the mammal an NK-1 antagonist wherein the NK-1 antagonist is rolapitant, or a pharmaceutically acceptable salt thereof. In some embodiments is a method of treating pruritus in a mammal in need thereof, the method comprising administering to the mammal a compound of Formula (I), or a pharmaceutically acceptable salt thereof, further comprising administering to the mammal an NK-1 antagonist wherein the NK-1 antagonist is aprepitant, or a pharmaceutically acceptable salt thereof. In some embodiments is a method of treating pruritus in a mammal in need thereof, the method comprising administering to the mammal a compound of Formula (I), or a pharmaceutically acceptable salt thereof, further comprising administering to the mammal an NK-1 antagonist wherein the NK-1 antagonist is fosaprepitant, or a pharmaceutically acceptable salt thereof.

In some embodiments of the pharmaceutical combinations described herein, the P2X3 antagonist is a compound of Formula (I), or a pharmaceutically acceptable salt thereof, and the NK-1 antagonist is a compound described in US2005/0176715, which are incorporated herein by reference.

In some embodiments of the pharmaceutical combinations described herein, the P2X3 antagonist is a compound of Formula (I), or a pharmaceutically acceptable salt thereof, and the NK-1 antagonist is a compound described in US2017/0326141, which are incorporated herein by reference.

Combination therapy also embraces the administration of the therapeutic agents as described above in further combination with other biologically active ingredients and non-drug therapies. Where the combination therapy further comprises a non-drug treatment, the non-drug treatment is conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as conventional oral dosage forms, that are administered either simultaneously or sequentially.

### EXAMPLES

This example is provided for illustrative purposes only and not to limit the scope of the claims provided herein.

### Example 1: Chloroquine-Induced Mouse Model of Acute Itch

The effect of a P2X3 receptor antagonist compound described herein in the chloroquine-induced mouse model of acute itch and its potentiation by α,β-methylene-adenosine 5′-triphosphate (α,β-Me-ATP) were evaluated. Compound 1 was evaluated at three doses. A κ-opiod agonist, U50,488, was used as a positive control.

Low dose chloroquine (CQ, 20 μg, Sigma) and α,β-Me-ATP (10, 50 or 100 μM) were dissolved in saline (0.9% NaCl). High dose CQ (200 μg) was dissolved in saline. Test article (2, 10 or 50 mg/kg body mass, calculated from 25 g body mass of each mouse) was dissolved in vehicle (saline with pH adjusted to 5.3 to completely dissolve). U50,488 (3 mg/kg, Sigma) was dissolved in vehicle (saline).

Mice were shaved at the nape and put into behavior chambers twice for 30 min to acclimate prior to injections and itch behavior. Mice were pre-injected intraperitoneally (i.p.) with vehicle, Compound 1, or U50,488 in a volume of 100 μL 30 min prior to intradermal injection. Mice were injected intradermally (i.d.) with compounds in a volume of 50 μL at the nape skin and placed individually into behavior chambers and video recorded at a side angle for 30 minutes. A scratch was defined as a lifting of the hind limb towards the nape or head to scratch and then a replacing of the limb back to the floor, regardless of how many scratching strokes take place between lifting and lowering of the hind limb (Munanairi et al. Cell Rep. 2018, 23, 866-877; Yu et al., Science 2017, 355, 1072-1076). Experimenters for injections and observers of scratching behavior were blinded to the injection compounds and groups of mice, respectively.

All data are presented as the mean number of scratches ±the standard error of the mean (s.e.m.). One-way ANOVA with Tukey multiple comparisons post-hoc test was performed for total scratching in 15 min for comparison of more than 2 groups. Unpaired t test was performed for total scratching in 15 or 30 min for comparison of 2 groups. Two-way RM ANOVA with Tukey or Sidak multiple comparisons post-hoc test was performed for time course in 5 min. intervals of scratching for all data sets.

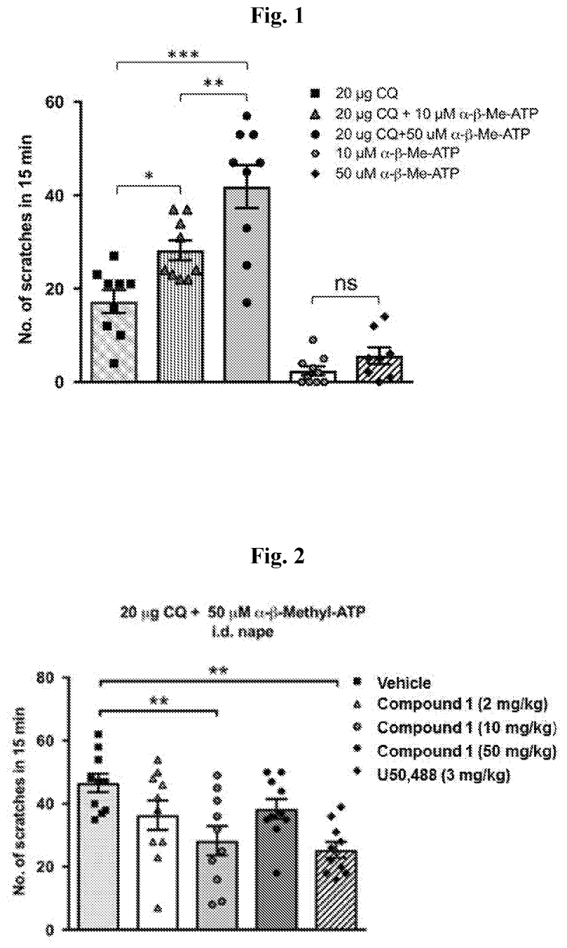

Compared to mice injected with CQ only, mice co-injected with CQ+10 μM α,β-Me-ATP showed increased scratching behavior, whereas mice injected solely with 10 μM α,β-Me-ATP showed almost no scratching (2.4±0.9 scratches). The increase in scratching with CQ+10 μM α,β-Me-ATP was statistically significant (p=0.0013, CQ vs CQ+10 μM α,β-Me-ATP) albeit a relatively modest increase (17.2±2.4 CQ vs 28.2±2.2 CQ+10 μM α,β-Me-ATP). To further test the potentiation effect of α,β-Me-ATP on CQ-induced itch, mice were co-injected with CQ+50 μM α,β-Me-ATP (FIG. 1). Compared to CQ only or CQ+10 μM α,β-Me-ATP, mice co-injected with CQ+50 μM α,β-Me-ATP showed further increased scratching behaviors. Mice injected solely with 50 μM α,β-Me-ATP showed little scratching behaviors (5.6±1.8) comparable to 10 μM α,β-Me-ATP. The increase in scratching with CQ+50 μM α,β-Me-ATP was statistically significant (p<0.001, CQ vs CQ+50 μM α,β-Me-ATP)

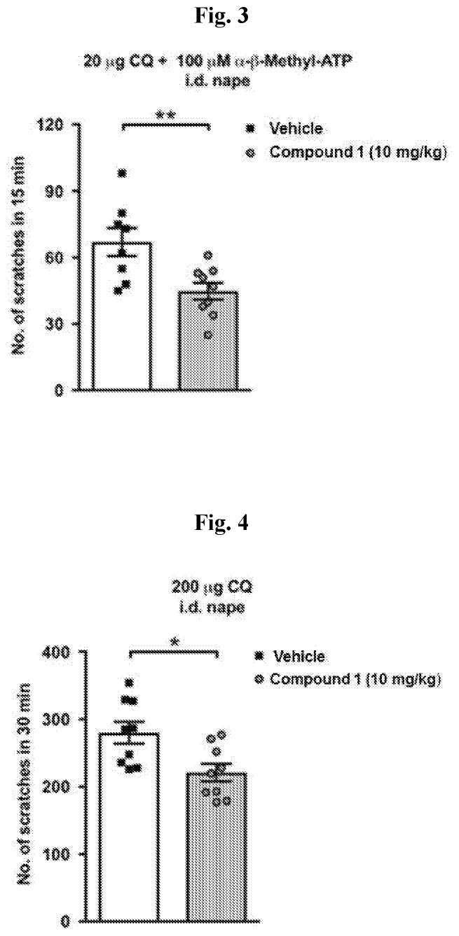

Next, the effect of the test article on acute itch was tested. Compared to mice pre-injected with vehicle, mice pre-injected with test article at 2, 10, or 50 mg/kg showed decreased scratching behavior for 20 μg CQ+50 μM α,β-Me-ATP, with 10 mg/kg dose showing a significant difference compared to vehicle (p=0.0084, 46.6±2.9 scratches for vehicle vs. 28.3±4.6 scratches for test article 10 mg/kg) (FIG. 2). The effect was similar to U50,488 on decreasing scratching behavior (p=0.0016, 46.6±2.9 scratches for vehicle vs. 25.4±2.5 scratches for U50,488 3 mg/kg). Next a higher concentration of α,β-Me-ATP was tested with CQ itch and the test article at 10 mg/kg was pre-injected. Compared to mice pre-injected with vehicle, mice pre-injected with test article at 10 mg/kg also showed a significant difference in total scratching behavior for 20 μg CQ+100 μM α,β-Me-ATP (p=0.0074, 67±6.3 scratches for vehicle vs. 44.8±3.8 scratches for test article 10 mg/kg) (FIG. 3). Lastly, a high dose of CQ (200 μg) was tested and the test article at 10 mg/kg was pre-injected. Compared to mice pre-injected with vehicle, mice pre-injected with test article at 10 mg/kg showed a significant difference in total scratching behavior for 200 μg CQ (p=0.011, 280±16.1 scratches for vehicle vs. 221.8±12.9 scratches for test article 10 mg/kg) (FIG. 4).

Taken together, the data indicates that ATP can effectively potentiate CQ-induced itch. However, ATP does not show apparent activity as a pruritogenper se when injected alone at the concentrations tested in this study. The data also indicates that Compound 1 effectively inhibited (40% decrease) the potentiation of CQ-induced itch by ATP, similar to the inhibitory effect (45% decrease) of the KOR agonist U50,488. Moreover, Compound 1 was also effective at inhibiting (33% decrease) the potentiation of CQ-induced itch by higher concentrations of ATP. Finally, the test article was effective (21% decrease) at inhibiting itch induced by high dose CQ.

### Example 2: AEW (Acetone-Ether-Water) Dry Skin Model

The nape of C57B16/J male mice (6 weeks old) was shaved and a mixture of acetone and diethyl ether (1:1) was applied with a cotton pad on the nape skin for 15 seconds, followed immediately by a 30 second distilled water application. This regimen was administered twice daily for 9 days. On Day 10, mice were pre-injected intraperitoneally with vehicle, Compound 1 (2, 10, or 50 mg/kg), or U50,488 (3 mg/kg) in a volume of 4 mL/kg body mass 30 min prior to monitoring of itch behaviors. Mice were placed individually into behavior chambers and video recorded at a side angle for 60-90 minutes. A scratch was defined as a lifting of the hind limb towards the nape or head to scratch and then a replacing of the limb back to the floor, regardless of how many scratching strokes take place between lifting and lowering of the hind limb. Experimenters for injections and observers of scratching behavior were blinded to the injection compounds and groups of mice, respectively.

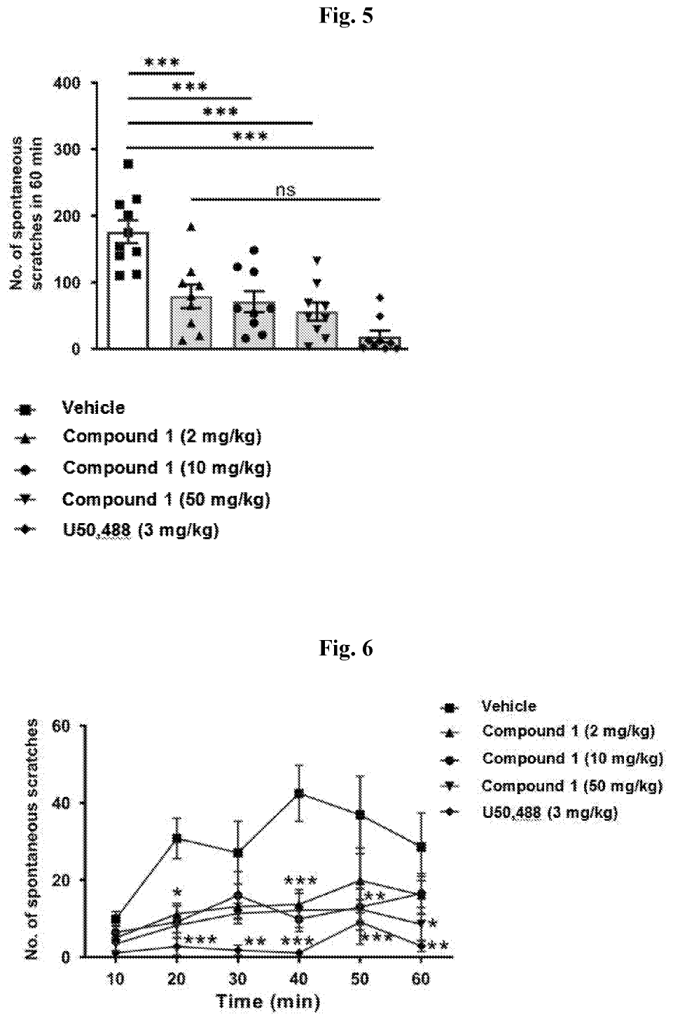

Compared to mice injected with vehicle, mice injected with 2, 10, or 50 mg/kg of Compound 1 showed decreased spontaneous scratches in 60 min, similar to U50,488-injected mice (positive control) (***p<0.001; ns: not significant) (FIG. 5). The decrease in scratching was statistically significant for all Compound 1 doses compared to vehicle. Time course analysis in 10-min intervals indicated that Compound 1 showed a significant effect on spontaneous scratches at 20 min, 40 min, 50 min, and 60 min (*p<0.05; **p<0.01; ***p<0.001) (FIG. 6).

### Example 3: Atopic Dermatitis Model

MC903 (calcipotriol, Tocris) was dissolved in 100% ethanol and topically applied on C57B16/J male mouse ears (4 nmol in 40 μl, 10 μl per side of ear) or nape (4 nmol in 40 μl). This regimen was administered twice daily for 7 days. Scratching behaviors were recorded 16 h after the last MC903 treatment. On Day 8, mice were pre-injected intraperitoneally with vehicle, Compound 1 (2, 10, or 50 mg/kg), or U50,488 (3 mg/kg) in a volume of 4 mL/kg body mass 30 min prior to monitoring of itch behaviors. Mice were placed individually into behavior chambers and video recorded at a side angle for 60-90 minutes. A scratch was defined as a lifting of the hind limb towards the nape or head to scratch and then a replacing of the limb back to the floor, regardless of how many scratching strokes take place between lifting and lowering of the hind limb. Experimenters for injections and observers of scratching behavior were blinded to the injection compounds and groups of mice, respectively.

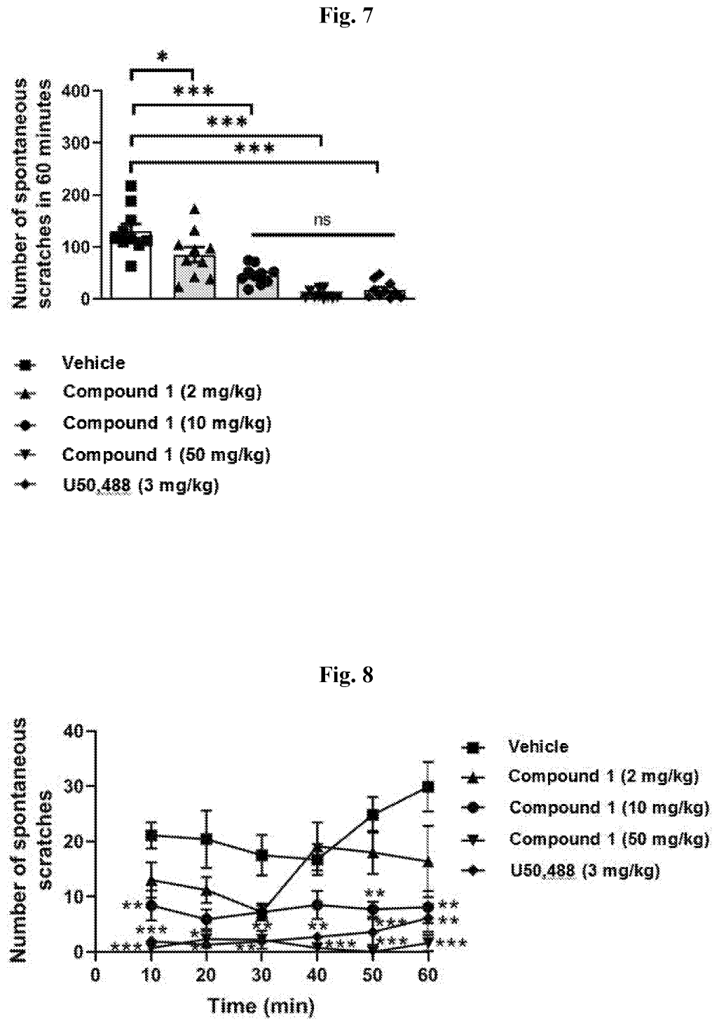

Compared to mice injected with vehicle, mice injected with 2, 10, or 50 mg/kg of Compound 1 showed dose-dependently decreased spontaneous scratches in 60 min. The decrease in scratching was statistically significant for all three doses. The two high doses of Compound 1 (10 mg/kg and 50 mg/kg) generated similar effect as the positive control (U50,488) (*p<0.05; ***p<0.001; ns: not significant) (FIG. 7). Time course analysis in 10-min intervals indicated that the test article showed a significant effect on spontaneous scratches at 10 min, 20 min, 30 min, 40 min, 50 min, and 60 min time points (*p<0.05; **p<0.01; ***p<0.001) (FIG. 8).

We claim:

1. A method of treating pruritus in a mammal, the method comprising administering to the mammal a therapeutically effective amount of a P2X3 antagonist.

2. The method of claim 1, wherein the P2X3 antagonist is a compound of Formula (I), or a pharmaceutically acceptable salt thereof, wherein Formula (I) is: 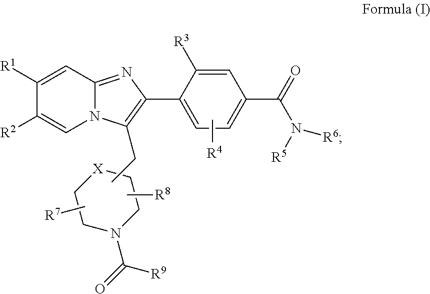
R¹ is selected from the group consisting of cyano, halogen, methyl, and ethyl;
R² is selected from the group consisting of hydrogen, halogen, methyl, and ethyl;
R³ is selected from the group consisting of halogen, methyl, and ethyl;
R⁴ is selected from the group consisting of hydrogen, halogen, methyl, ethyl, and methoxy;
R⁵ and R⁶ are independently selected from the group consisting of hydrogen, C₁-C₆-alkyl, and hydroxy-C₁-C₆-alkyl; or
R⁵ and R⁶, together with the nitrogen to which they are both attached, form a 5- or 6-member heterocycloalkyl, wherein the heterocycloalkyl is optionally substituted with one or more substituents independently selected from the group consisting of halogen, hydroxyl, and C₁-C₄-alkyl;
R⁷ and R⁸ are independently selected from the group consisting of hydrogen and C₁-C₄-alkyl;
R⁹ is selected from the group consisting of C₁-C₆-alkyl, C₃-C₆-cycloalkyl, C₁-C₆-alkyl-C₃-C₆-cycloalkyl, halo-C₁-C₆-alkyl, C₁-C₆-alkoxy, halo-C₁-C₆-alkoxy, and C₁-C₆-alkoxy-C₁-C₆-alkyl; and
X is selected from the group consisting of a bond, CH₂, and O.

3. The method of claim 2, wherein R¹ is methyl and R² is hydrogen.

4. The method of claim 2, wherein R³ and R⁴ are fluoro.

5. The method of claim 2, wherein X is O.

6. The method of claim 2, wherein the compound corresponds in structure to: 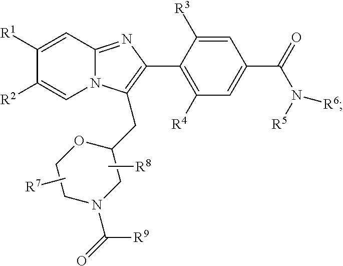
R⁴ is selected from the group consisting of halogen, methyl, and ethyl.

7. The method of claim 2, wherein R⁵ is hydrogen and R⁶ is C₁-C₆-alkyl.

8. The method of claim 2, wherein R⁶ is methyl.

9. The method of claim 2, wherein R⁷ and R⁸ are hydrogen.

10. The method of claim 2, wherein R⁹ is C₁-C₆-alkoxy.

11. The method of claim 2, wherein R⁹ is methoxy.

12. The method of claim 2, wherein the compound corresponds in structure to:

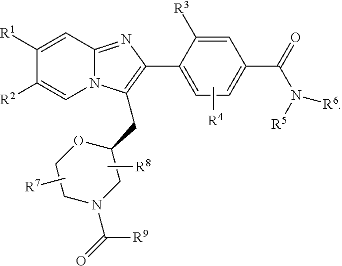

13. The method of claim 2, wherein the compound corresponds in structure to:

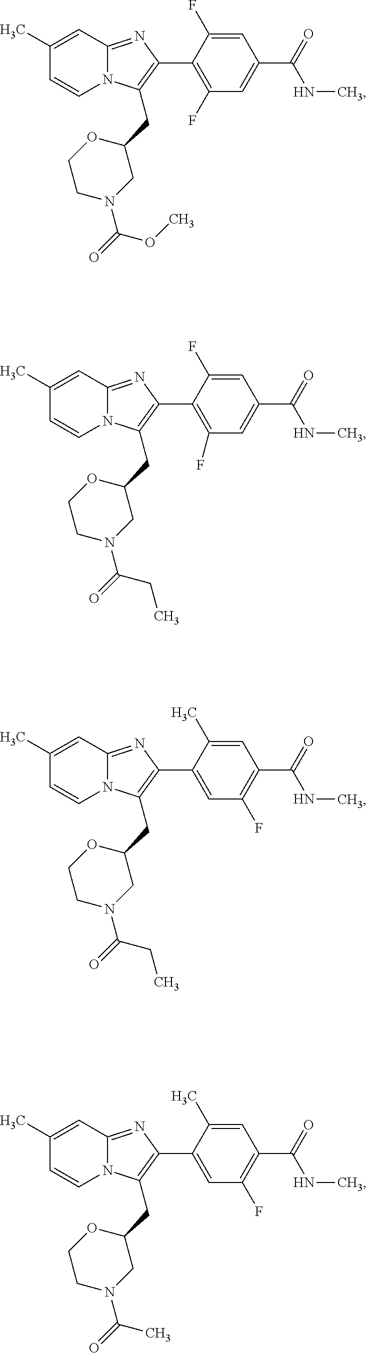 [SMILES: CNC(=O)c1cc(F)c(-c2nc3cc(C)ccn3c2C[C@H]2CN(C(=O)OC)CCO2)c(F)c1 ; CCC(=O)N1CCO[C@@H](Cc2c(-c3c(F)cc(C(=O)NC)cc3F)nc3cc(C)ccn23)C1 ; CCC(=O)N1CCO[C@@H](Cc2c(-c3cc(F)c(C(=O)NC)cc3C)nc3cc(C)ccn23)C1 ; CNC(=O)c1cc(C)c(-c2nc3cc(C)ccn3c2C[C@H]2CN(C(C)=O)CCO2)cc1F]

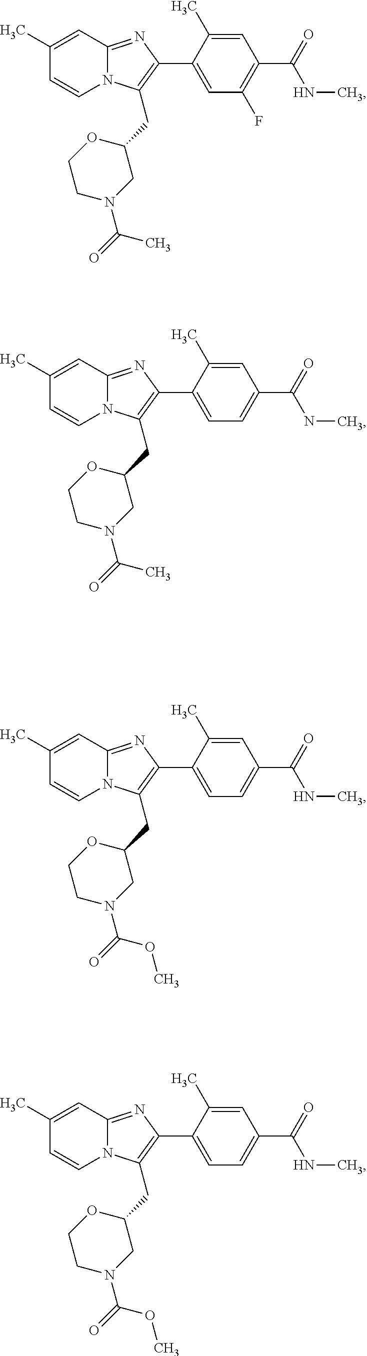 [SMILES: CNC(=O)c1cc(C)c(-c2nc3cc(C)ccn3c2C[C@@H]2CN(C(C)=O)CCO2)cc1F ; CNC(=O)c1ccc(-c2nc3cc(C)ccn3c2C[C@H]2CN(C(C)=O)CCO2)c(C)c1 ; CNC(=O)c1ccc(-c2nc3cc(C)ccn3c2C[C@H]2CN(C(=O)OC)CCO2)c(C)c1 ; CNC(=O)c1ccc(-c2nc3cc(C)ccn3c2C[C@@H]2CN(C(=O)OC)CCO2)c(C)c1]

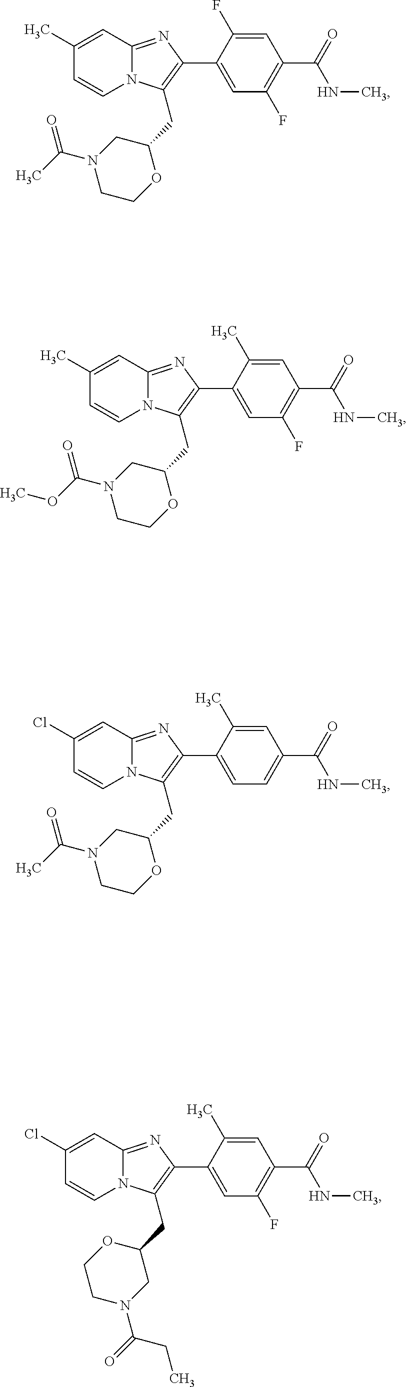 [SMILES: CNC(=O)c1cc(F)c(-c2nc3cc(C)ccn3c2C[C@H]2CN(C(C)=O)CCO2)cc1F ; CNC(=O)c1cc(C)c(-c2nc3cc(C)ccn3c2C[C@H]2CN(C(=O)OC)CCO2)cc1F ; CNC(=O)c1ccc(-c2nc3cc(Cl)ccn3c2C[C@H]2CN(C(C)=O)CCO2)c(C)c1 ; CCC(=O)N1CCO[C@@H](Cc2c(-c3cc(F)c(C(=O)NC)cc3C)nc3cc(Cl)ccn23)C1]

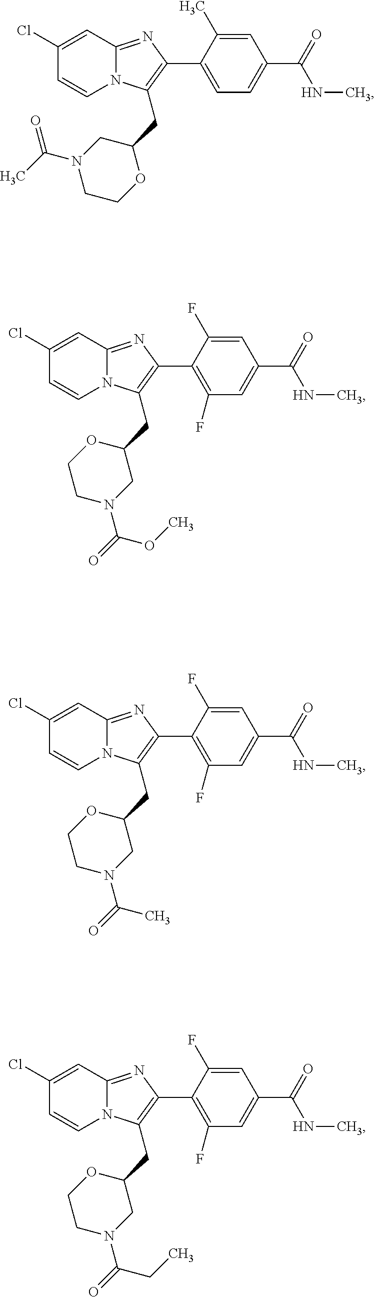 [SMILES: CNC(=O)c1ccc(-c2nc3cc(Cl)ccn3c2C[C@@H]2CN(C(C)=O)CCO2)c(C)c1 ; CNC(=O)c1cc(F)c(-c2nc3cc(Cl)ccn3c2C[C@H]2CN(C(=O)OC)CCO2)c(F)c1 ; CNC(=O)c1cc(F)c(-c2nc3cc(Cl)ccn3c2C[C@H]2CN(C(C)=O)CCO2)c(F)c1 ; CCC(=O)N1CCO[C@@H](Cc2c(-c3c(F)cc(C(=O)NC)cc3F)nc3cc(Cl)ccn23)C1]

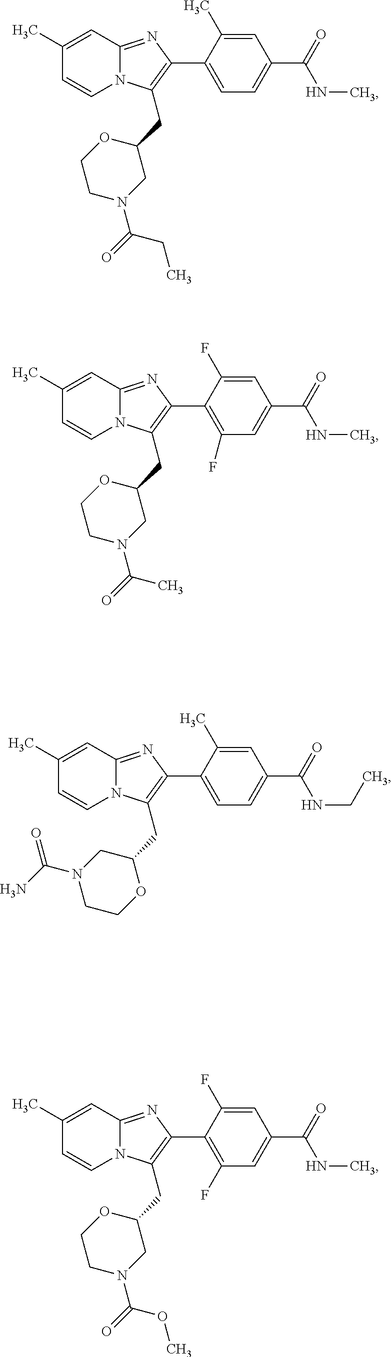 [SMILES: CCC(=O)N1CCO[C@@H](Cc2c(-c3ccc(C(=O)NC)cc3C)nc3cc(C)ccn23)C1 ; CNC(=O)c1cc(F)c(-c2nc3cc(C)ccn3c2C[C@H]2CN(C(C)=O)CCO2)c(F)c1 ; CCNC(=O)c1ccc(-c2nc3cc(C)ccn3c2C[C@H]2CN(C(N)=O)CCO2)c(C)c1 ; CNC(=O)c1cc(F)c(-c2nc3cc(C)ccn3c2C[C@@H]2CN(C(=O)OC)CCO2)c(F)c1]

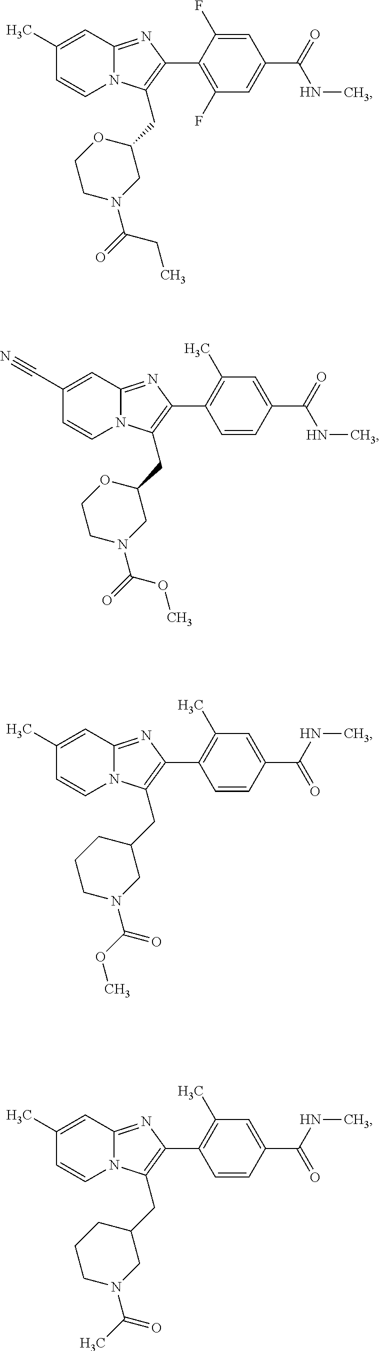 [SMILES: CCC(=O)N1CCO[C@H](Cc2c(-c3c(F)cc(C(=O)NC)cc3F)nc3cc(C)ccn23)C1 ; CNC(=O)c1ccc(-c2nc3cc(C#N)ccn3c2C[C@H]2CN(C(=O)OC)CCO2)c(C)c1 ; CNC(=O)c1ccc(-c2nc3cc(C)ccn3c2CC2CCCN(C(=O)OC)C2)c(C)c1 ; CNC(=O)c1ccc(-c2nc3cc(C)ccn3c2CC2CCCN(C(C)=O)C2)c(C)c1]

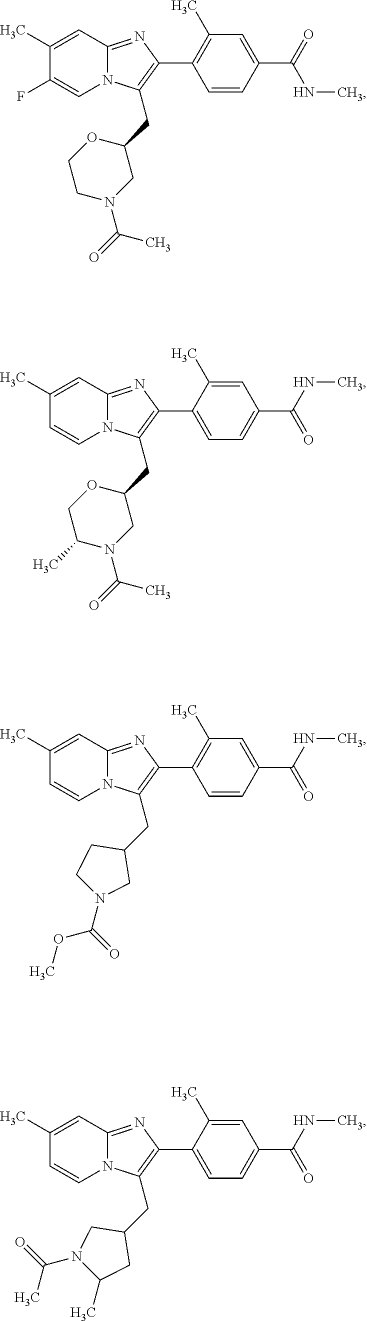 [SMILES: CNC(=O)c1ccc(-c2nc3cc(C)c(F)cn3c2C[C@H]2CN(C(C)=O)CCO2)c(C)c1 ; CNC(=O)c1ccc(-c2nc3cc(C)ccn3c2C[C@H]2CN(C(C)=O)[C@H](C)CO2)c(C)c1 ; CNC(=O)c1ccc(-c2nc3cc(C)ccn3c2CC2CCN(C(=O)OC)C2)c(C)c1 ; CNC(=O)c1ccc(-c2nc3cc(C)ccn3c2CC2CC(C)N(C(C)=O)C2)c(C)c1]

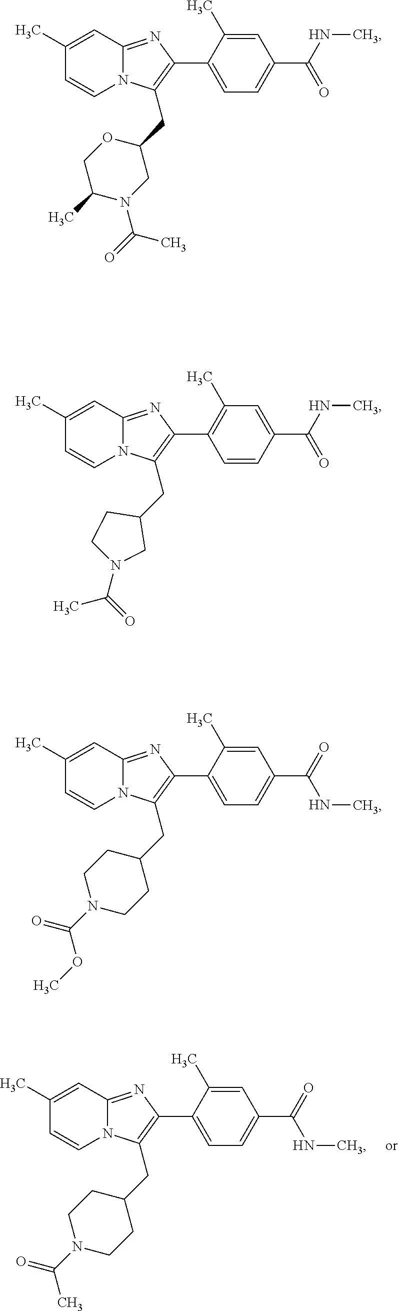 [SMILES: CNC(=O)c1ccc(-c2nc3cc(C)ccn3c2C[C@H]2CN(C(C)=O)[C@@H](C)CO2)c(C)c1 ; CNC(=O)c1ccc(-c2nc3cc(C)ccn3c2CC2CCN(C(C)=O)C2)c(C)c1 ; CNC(=O)c1ccc(-c2nc3cc(C)ccn3c2CC2CCN(C(=O)OC)CC2)c(C)c1 ; CNC(=O)c1ccc(-c2nc3cc(C)ccn3c2CC2CCN(C(C)=O)CC2)c(C)c1]

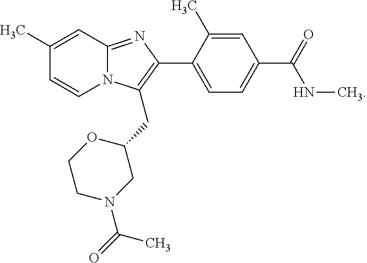 [SMILES: CNC(=O)c1ccc(-c2nc3cc(C)ccn3c2C[C@@H]2CN(C(C)=O)CCO2)c(C)c1]

14. The method of claim 2, wherein the compound corresponds in structure to:

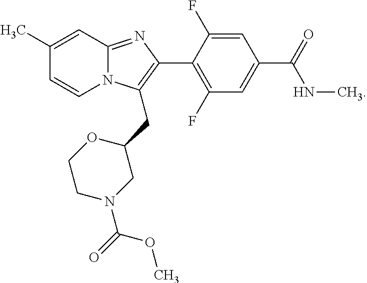 [SMILES: CNC(=O)c1cc(F)c(-c2nc3cc(C)ccn3c2C[C@H]2CN(C(=O)OC)CCO2)c(F)c1]

15. The method of claim 2, wherein the compound corresponds in structure to:

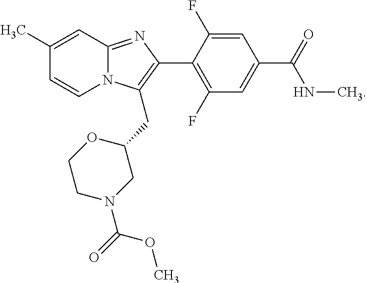 [SMILES: CNC(=O)c1cc(F)c(-c2nc3cc(C)ccn3c2C[C@@H]2CN(C(=O)OC)CCO2)c(F)c1]

16. The method of claim 2, wherein the compound corresponds in structure to:

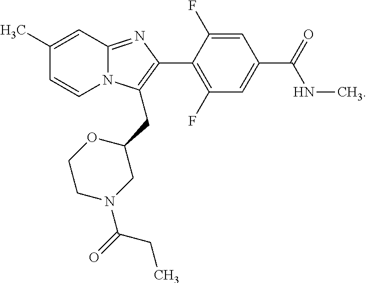 [SMILES: CCC(=O)N1CCO[C@@H](Cc2c(-c3c(F)cc(C(=O)NC)cc3F)nc3cc(C)ccn23)C1]

17. The method of claim 2, wherein the compound corresponds in structure to:

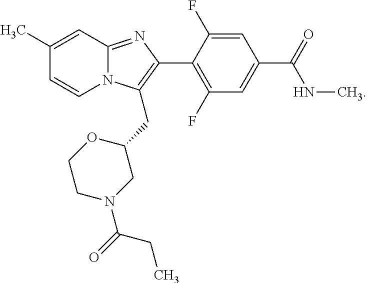 [SMILES: CCC(=O)N1CCO[C@H](Cc2c(-c3c(F)cc(C(=O)NC)cc3F)nc3cc(C)ccn23)C1]

18. The method of claim 1, wherein the P2X3 antagonist corresponds in structure to:

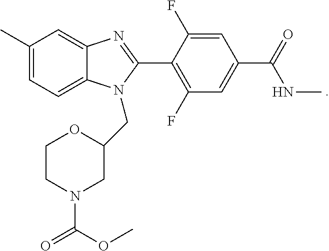

19. The method of claim 1, wherein the P2X3 antagonist corresponds in structure to:

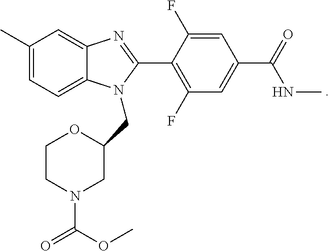

20. The method of claim 1, wherein the P2X3 antagonist corresponds in structure to:

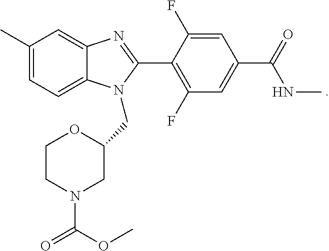

21. The method of any one of claims 1-20, wherein the mammal is a human.

22. The method of any one of claims 1-21, wherein the pruritus is associated with an inflammatory skin disease, an infectious skin disease, an autoimmune skin disease, or a pregnancy-related skin disease.

23. The method of claim 22, wherein the pruritus is associated with an inflammatory skin disease selected from the group consisting of atopic dermatitis, allergic, irritant contact dermatitis, exsiccation dermatitis, nummular and dyshidrotic dermatitis, lichen planus, lichen sclerosus et atrophicus, polymorphous light eruption psoriasis, Grover's disease, mucinosis, mastocytosis, and urticaria.

24. The method of claim 22, wherein the pruritus is associated with an infectious skin disease selected from the group consisting of mycoses, bacterial and viral infections, scabies, pediculosis, insect bites, and folliculitides.

25. The method of claim 22, wherein the pruritus is associated with an autoimmune skin disease selected from the group consisting of dermatitis herpetiformis (Duhring's disease), bullous pemphigoid; genodermatoses, Darier's disease, and Hailey-Hailey disease.

26. The method of claim 22, wherein the pruritus is associated with a pregnancy-related skin disease selected from the group consisting of polymorphic eruption of pregnancy (PEP), atopic eruption of pregnancy, pemphigoid gestationis, neoplasias, and cutaneous T-cell lymphoma.

27. The method of any one of claims 1-21, wherein the pruritus is associated with prurigo nodularis.

28. The method of any one of claims 1-21, wherein the pruritus is associated with a kidney disease or a therapeutic procedure to treat a kidney disease.

29. The method of claim 28, wherein the pruritus is associated with a chronic kidney disease.

30. The method of claim 28, wherein the pruritus is associated with a therapeutic procedure to treat a kidney disease, wherein the therapeutic procedure to treat the kidney disease is selected from the group consisting of hemodialysis and peritoneal dialysis.

31. The method of any one of claims 1-21, wherein the pruritus is associated with a medical procedure or treatment.

32. The method of claim 31, wherein the pruritus is associated with a medical treatment with a drug selected from the group consisting of opioids, anti-malarial drugs, anti-cancer therapies and epidermal growth factor receptor inhibitors.

33. The method of any one of claims 1-32, wherein the P2X3 antagonist is formulated for administration to a mammal by intravenous administration, subcutaneous administration, oral administration, inhalation, nasal administration, topical administration, or ophthalmic administration.

34. The method of claim 33, wherein the pharmaceutical composition is in the form of a tablet, a pill, a capsule, a liquid, a suspension, a gel, a dispersion, a solution, an emulsion, an ointment, or a lotion.

35. The method of any one of claims 1-34, further comprising the administration of a second therapeutic agent.

36. The method of any one of claims 1-35, further comprising the administration of a NK-1 antagonist.

37. The method of claim 36, wherein the NK-1 antagonist is selected from the group consisting of serlopitant, aprepitant, casopitant, dapitant, ezlopitant, fosaprepitant, lanepitant, maropitant, netupitant, nolpitant, orvepitant, rolapitant, vestipitant, vofopitant, AV-818, BIIF 1149CL, CP122,721, DNK-333, GSK-424887, L-733060, L-759274, LY-686017, M516102, and TA-5538.
